(19) United States
(12) Patent Application Publication
(10) Pub. No.: US 2006/0235013 A1
(43) Pub. Date: Oct. 19, 2006

(54) Tricyclic azole derivatives

(76) Inventors: Guy Georges, Habach (DE); Bernhard Goller, Penzberg (DE); Hans-Willi Krell, Penzberg (DE); Anja Limberg, Penzberg (DE); Ulrike Reiff, Penzberg (DE); Petra Rueger, Penzberg (DE); Matthias Rueth, Penzberg (DE)

Correspondence Address: HOFFMANN-LA ROCHE INC.;PATENT LAW DEPARTMENT, 340 KINGSLAND STREET, NUTLEY, NJ, 07110, US

(21) Appl. No.: 11/384,058

(22) Filed: Mar. 17, 2006

(30) Foreign Application Priority Data
Apr. 14, 2005 (EP) 05 008 112.4

(51) Int. Cl. A61K 31/5377; A61K 31/4745; A61K 31/4188; C07D 487/04

(52) U.S. Cl. 514/234.2; 514/393; 544/132; 514/292; 546/82; 548/302.1

(57) ABSTRACT

The present invention relates to the compounds of formula I: 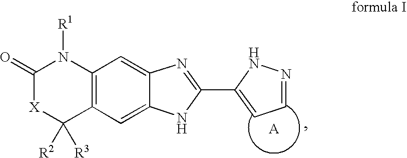 their pharmaceutically acceptable salts or esters, enantiomeric forms, diastereoisomers and racemates, the preparation of the above-mentioned compounds, pharmaceutical compositions containing them and their manufacture, as well as the use of such compounds in the control or prevention of illnesses such as cancer.

### PRIORITY TO RELATED APPLICATIONS

This application claims the benefit of European Application No. 05008112.4, filed Apr. 14, 2005, which is hereby incorporated by reference in its entirety.

### FIELD OF THE INVENTION

The present invention relates to novel tricyclic azole derivatives, to a process for their manufacture, pharmaceutical compositions containing them and their manufacture as well as the use of these compounds as pharmaceutically active agents.

### BACKGROUND OF THE INVENTION

Protein kinases regulate many different signaling processes by adding phosphate groups to proteins (Hunter, T., Cell 50 (1987) 823-829); particularly serine/threonine kinases phosphorylate proteins on the alcohol moiety of serine or threonine residues. The serine/threonine kinase family includes members that control cell growth, migration, differentiation, gene expression, muscle contraction, glucose metabolism, cellular protein synthesis, and regulation of the cell cycle.

Many diseases are associated with abnormal cellular responses triggered by protein kinase mediated events. These diseases include autoimmune diseases, inflammatory diseases, neurological and neurodegenerative diseases, cancer, cardiovascular diseases, allergies and asthma, Alzheimer's disease or hormone-related diseases. Accordingly, there has been a substantial effort in medicinal chemistry to find protein kinase inhibitors that are effective as therapeutic agents.

The Aurora kinases are a family of serine/threonine kinases that are believed to play a key role in the protein phosphorylation events that are essential for the completion of essential mitotic events. The Aurora kinase family is made up of three key members: Aurora A, B and C (also known as Aurora-2, Aurora-1 and Aurora-3 respectively). Aurora-1 and Aurora-2 are described in U.S. Pat. No. 6,207,401 of Sugen and in related patents and patent applications, e.g. EP 0 868 519 and EP 1 051 500.

For Aurora A there is increasing evidence that it is a novel proto-oncogene. The Aurora A gene is amplified and the transcript/protein is highly expressed in a majority of human tumor cell lines and primary colorectal, breast and other tumors. It has been shown that Aurora A overexpression leads to genetic instability shown by amplified centrosomes and significant increase in aneuploidy and transforms Rat1 fibroblasts and mouse NIH3T3 cells in vitro. Aurora A-transformed NIH3T3 cells grow as tumors in nude mice (Bischoff, J. R., and Plowman, G. D., Trends Cell Biol. 9 (1999) 454-459; Giet, R., and Prigent, C., J. Cell Sci. 112 (1999) 3591-3601; Nigg, E. A., Nat. Rev. Mol. Cell Biol. 2 (2001) 21-32; Adams, R. R., et al., Trends Cell Biol. 11 (2001) 49-54). Moreover, amplification of Aurora A is associated with aneuploidy and aggressive clinical behavior (Sen, S., et al., J. Natl. Cancer Inst. 94 (2002) 1320-1329) and amplification of its locus correlates with poor prognosis for patients with node-negative breast cancer (Isola, J. J., et al., Am. J. Pathology 147 (1995) 905-911). For these reasons it is proposed that Aurora A overexpression contributes to cancer phenotype by being involved in chromosome segregation and mitotic checkpoint control.

Human tumor cell lines depleted of Aurora A transcripts arrest in mitosis. Accordingly, the specific inhibition of Aurora kinase by selective inhibitors is recognized to stop uncontrolled proliferation, re-establish mitotic checkpoint control and lead to apoptosis of tumor cells. In a xenograft model, an Aurora inhibitor therefore slows tumor growth and induces regression (Harrington, E. A., et al., Nat. Med. 10 (2004) 262-267).

Low molecular weight inhibitors for protein kinases are widely known in the state of the art. For Aurora inhibition such inhibitors are based on for example: quinazoline derivatives as disclosed in WO 00/44728; WO 00/47212; WO 01/21594; WO 01/21595; WO 01/21596; WO 01/21597; WO 01/77085; WO 01/55116; WO 95/19169; WO 95/23141; WO 97/42187; and WO 99/06396; pyrazole and triazole derivatives as disclosed in WO 02/22601; WO 02/22602; WO 02/22603; WO 02/22604; WO 02/22605; WO 02/22606; WO 02/22607; WO 02/22608; WO 02/50065; WO 02/50066; WO 02/057259; WO 02/059112; WO 02/059111; WO 02/062789; WO 02/066461; and WO 02/068415; pyrimidine derivatives as disclosed in WO 03/077921; WO 03/078423; WO 03/078426; WO 03/078427; and WO 04/000833; and imidazole, oxazole and thiazole derivatives as disclosed in WO 02/96905 and WO 04/005283.

JP 03/231687 relates to condensed pyrazole derivatives as neutrophin-inhibiting and analgetic agents. WO 03/035065 relates to benzimidazole derivatives as kinase inhibitors, especially as inhibitors against kinase insert domain containing receptor (KDR) tyrosine kinase, spleen tyrosine kinase (SYK) and inducible T cell kinase (ITK). And WO 2005/007653 refers to substituted 4,5,6,7-tetrahydropyrazolo[3,4-c]pyridines as kinase inhibitors, especially as inhibitors against tyrosine kinase with immunoglobulin and EGF (epidermal growth factor) repeats 2 (Tie 2) and KDR tyrosine kinase.

Some related tricyclic compounds are known as inhibitors of erythrocyte aggregation from U.S. Pat. No. 4,835,280A and U.S. Pat. No. 4,954,498A. Also Mertens, A., et al., J. Med. Chem. 30 (1987) 1279-1287; von der Saal, W., et al., J. Med. Chem. 32 (1989) 1481-1491; U.S. Pat. No. 4,666,923A; U.S. Pat. No. 4,695,567A and U.S. Pat. No. 4,863,945A describe related tricycles as erythrocyte aggregation inhibitors. U.S. Pat. No. 5,212,186A describes related tricycles for the treatment of cardiac insuffiency, hypertension and other diseases. WO 2005/111040 describes pyrrolobenzimidazolones with tubulin inhibitory activity as antiproliferative agents.

### SUMMARY OF THE INVENTION

The present invention relates to tricyclic azole derivatives of the general formula I and all pharmaceutically acceptable salts or esters thereof wherein formula I is: 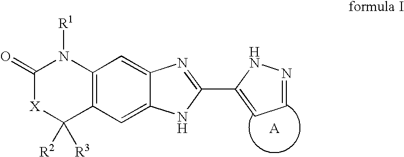
wherein:
(a) R¹ is selected from the group consisting of:
   (1) hydrogen;
   (2) alkyl, which is optionally substituted one or more times with nitro, cyano or —Y—R⁴;
   (3) alkenyl, which is optionally substituted one or more times with nitro, cyano or —Y—R⁴; and
   (4) alkynyl, which is optionally substituted one or more times with nitro, cyano or —Y—R⁴;
(b) Y is selected from the group consisting of:
   (1) a single bond,
   (2) —C(O)NH—;
   (3) —C(O)N(alkyl)-;
   (4) —N(alkyl)C(O)—;
   (5) —NHC(O)—;
   (6) —NHC(O)NH—;
   (7) —NHC(O)N(alkyl)-;
   (8) —NHS(O)₂—;
   (9) —S(O)₂NH—;
   (10) —S(O)₂N(alkyl)-;
   (11) —S(O)₂—;
   (12) —S(O)—;
   (13) —C(O)O—;
   (14) —OC(O)—;
   (15) —C(O)—;
   (16) —P(O)(alkyl)-;
   (17) —NH—;
   (18) —N(alkyl)-;
   (19) —O—; and
   (20) —S—;
(c) R⁴ is selected from the group consisting of:
   (1) alkyl, which is optionally substituted one or more times by halogen, hydroxy, alkoxy, alkoxyalkoxy, amino, alkylamino, dialkylamino, —C(O)OH or —C(O)NH₂;
   (2) aryl, which is optionally substituted one or more times by halogen, cyano, nitro, amino, hydroxy, (C₁-C₄)alkyl, (C₁-C₄)alkoxy, halogenated (C₁-C₄)alkyl, or halogenated (C₁-C₄)alkoxy;
   (3) heteroaryl, which is optionally substituted one or more times by alkyl;
   (4) cycloalkyl; and
   (5) heterocyclyl;
(d) R² and R³ form together with the carbon atom to which they are attached a (C₅-C₆)cycloalkyl ring, or alternatively, R² and R³ are independently selected from the group consisting of:
   (1) hydrogen; and
   (2) alkyl;
(e) X is a single bond, —CH₂— or —C(alkyl)₂—;
(f) ring A is a 5 to 7 membered saturated ring optionally substituted one or more times by alkyl and optionally containing one or two heteroatoms independently selected from the group consisting of oxygen, nitrogen and sulfur, with the remaining ring atoms being carbon atoms, wherein if said ring A contains one nitrogen, said nitrogen can be optionally substituted once by a substituent selected from the group consisting of:
   (1) —CH₂-phenyl;
   (2) —C(O)-alkyl;
   (3) —C(O)-cycloalkyl;
   (4) —C(O)-heterocyclyl;
   (5) —C(O)—(CH₂)ₙ-phenyl, wherein the phenyl is optionally substituted once or twice with halogen, alkyl, alkoxy, nitro, amino, alkylamino, dialkylamino, cyano, trifluoromethyl or trifluoromethoxy;
   (6) —C(O)—(CH₂)ₙ-heteroaryl;
   (7) —C(O)—NH-phenyl, wherein the phenyl is optionally substituted once or twice with halogen, alkyl, alkoxy, nitro, amino, alkylamino, dialkylamino, cyano, trifluoromethyl or trifluoromethoxy; and
   (8) —S(O)₂-phenyl, wherein the phenyl is optionally substituted once or twice with halogen, alkyl, alkoxy, nitro, amino, alkylamino, dialkylamino, cyano; trifluoromethyl or trifluoromethoxy; and
(g) n is 0, 1 or 2.

The compounds of the present invention show activity as protein kinase inhibitors, and therefore such compounds are useful for prevevting or treating diseases associated with abnormal cellular responses triggered by protein kinase mediated events. In particular, the compounds of the present invention show activity as Aurora family kinase inhibitors, especially as Aurora A kinase inhibitors, and may therefore be useful for the treatment of diseases mediated by said kinase. Aurora A inhibition leads to cell cycle arrest in the G2 phase of the cell cycle and exerts an antiproliferative effect in tumor cell lines. This indicates that Aurora A inhibitors may be useful in the treatment of hyperproliferative diseases such as cancer and in particular colorectal cancer, breast cancer, lung cancer, prostate cancer, pancreatic cancer, gastric cancer, bladder cancer, ovarian cancer, melanoma cancer, neuroblastoma cancer, cervical cancer, kidney or renal cancers, leukemias and lymphomas. Auroa A inhibitors may also be useful for the treatment of acute-myelogenous leukemia (AML, acute lymphocytic leukemia (ALL) and gastrointestinal stromal tumor (GIST).

The present invention provides compounds of formula I and their tautomers, pharmaceutically acceptable salts or esters, enantiomeric forms, diastereoisomers and racemates, their use as Aurora kinase inhibitors, the preparation of the above-mentioned compounds, compositions containing them and their manufacture as well as the use of the above-mentioned compounds in treatment, control or prevention of illnesses, especially the illnesses and disorders mentioned above or in the manufacture of corresponding pharmaceutical compositions.

### DETAILED DESCRIPTION OF THE INVENTION

The term “alkyl” as used herein means a saturated, straight-chain or branched-chain hydrocarbon containing from 1 to 6 carbon atoms, preferably 1 to 4 carbon atoms, such as methyl, ethyl, n-propyl, isopropyl, n-butyl, 2-butyl, and t-butyl.

The term “alkenyl” as used herein means an unsaturated straight-chain or branched aliphatic hydrocarbon group containing one double bond and having 2 to 6 carbon atoms, preferably 2 to 4 carbon atoms. Examples of such “alkenyl groups” are vinyl(ethenyl), allyl, isopropenyl, 1-propenyl, 2-methyl-1-propenyl, 1-butenyl, 2-butenyl, 3-butenyl, 2-ethyl-1-butenyl, 3-methyl-2-butenyl, 1-pentenyl, 2-pentenyl, 3-pentenyl, 4-pentenyl, 4-methyl-3-pentenyl, 1-hexenyl, 2-hexenyl, 3-hexenyl, 4-hexenyl and 5-hexenyl.

The term “alkynyl” as used herein means an unsaturated straight-chain or branched aliphatic hydrocarbon group containing one triple bond and having 2 to 6 carbon atoms, preferably 2 to 4 carbon atoms. Examples of such “alkynyl groups” are ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl and 5-hexynyl.

The term “alkoxy” as used herein means an alkyl-O— group wherein the alkyl is defined as above.

The term “alkylamino” as used herein means an alkyl-NH— group wherein the alkyl is defined as above.

The term “dialkylamino” as used herein means an (alkyl)₂N— group wherein the alkyl is defined as above.

The term “halogenated alkyl” as used herein means an alkyl group as defined above which is substituted one or more times by halogen, preferably by fluorine or chlorine, more preferably fluorine. Examples are difluoromethyl, trifluoromethyl, 2,2,2-trifluoroethyl, perfluorethyl, and the like, preferably trifluoromethyl.

The term “halogenated alkoxy” as used herein means an alkoxy group as defined above which is substituted one or more times by halogen, preferably by fluorine or chlorine, especially fluorine. Examples are difluoromethoxy, trifluoromethoxy, 2,2,2-trifluoroethoxy, perfluoroethoxy and the like, preferably trifluoromethoxy.

The term “halogen” means fluorine, chlorine, bromine or iodine, preferably fluorine, chlorine or bromine and more preferably fluorine or chlorine.

The term “cycloalkyl” means a monocyclic saturated hydrocarbon ring with 3 to 7 ring atoms, preferably 3 to 6 ring atoms. Such saturated carbocyclic groups can be optionally substituted one or several times, preferably one to three times by alkyl, more preferably one to two times. Preferably such saturated carbocyclic groups are unsubstituted. Examples of such saturated carbocyclic groups are cyclopropyl, cyclobutyl, cyclopentyl, cyclohexyl, cycloheptyl, 3-methyl-cyclopentyl, 3,3-dimethyl-cyclohexyl, 3-methyl-cyclohexyl, 2-methyl-cyclohexyl, preferably cyclopropyl.

The term “heterocyclyl” means a saturated, monocyclic ring with 5 to 6 ring atoms which contains up to 3 heteroatoms, preferably 1 or 2 heteroatoms selected independently from the group consisting of N, O and S, with the remaining ring atoms being carbon atoms. Such a saturated heterocyclic group can be optionally substituted one or more times, preferably one or two times by a substituent selected from the group consisting of: (a) alkyl, preferably methyl, (b) —C(O)-alkyl, preferably acetyl, (c) oxo, and (d) —S(O)₂-alkyl. Preferred substituents are (a) alkyl or (b) —C(O)-alkyl. Examples of such saturated heterocyclic groups include pyrrolidinyl, morpholinyl, piperazinyl, N-methyl-piperazinyl, N-acetyl-piperazinyl, piperazin-2-one, piperidyl and the like, preferably morpholino (4-morpholinyl).

The term “aryl” means a mono- or bicyclic aromatic ring with 6 to 10 ring carbon atoms. Examples of such aryl groups are phenyl and naphthyl, preferably phenyl.

The term “heteroaryl” means a mono- or bicyclic aromatic ring with 5 to 10 ring atoms, preferably 5 to 6 ring atoms, which contains up to 3 heteroatoms, preferably 1 or 2 heteroatoms selected independently from the group consisting of: N, O and S; and the remaining ring atoms being carbon atoms. Examples of such heteroaryl groups include pyrrolyl, imidazolyl, pyrazolyl, triazolyl, tetrazolyl, furanyl, oxazolyl, isoxazolyl, thienyl, thiazolyl, pyridyl, pyrimidyl, pyridazinyl, pyrazinyl, indolyl, indazolyl, benzimidazolyl, benzothiophenyl, benzofuranyl, quinolyl, isoquinolyl, quinazolinyl and the like, preferably pyridyl.

As used herein, in relation to mass spectrometry (MS) the term “API+” refers to positive atmospheric pressure ionization mode.

As used herein, in relation to nuclear magnetic resonance (NMR) the term “D₆-DMSO” refers to deuterated dimethylsulfoxide.

As used herein, the term “a therapeutically effective amount” of a compound means an amount of compound that is effective to prevent, alleviate or ameliorate symptoms of disease or prolong the survival of the subject being treated. Determination of a therapeutically effective amount is within the skill in the art.

The therapeutically effective amount or dosage of a compound according to this invention can vary within wide limits and may be determined in a manner known in the art. Such dosage will be adjusted to the individual requirements in each particular case including the specific compound(s) being administered, the route of administration, the condition being treated, as well as the patient being treated. In general, in the case of oral or parenteral administration to adult humans weighing approximately 70 Kg, a daily dosage of about 10 mg to about 10,000 mg, preferably from about 200 mg to about 1,000 mg, should be appropriate, although the upper limit may be exceeded when indicated. The daily dosage can be administered as a single dose or in divided doses, or for parenteral administration, it may be given as continuous infusion.

As used herein, a “pharmaceutically acceptable carrier” is intended to include any and all material compatible with pharmaceutical administration including solvents, dispersion media, coatings, antibacterial and antifungal agents, isotonic and absorption delaying agents, and other materials and compounds compatible with pharmaceutical administration. Except insofar as any conventional media or agent is incompatible with the active compound, use thereof in the compositions of the invention are contemplated. Supplementary active compounds can also be incorporated into the compositions.

The compounds according to the present invention may exist in the form of their pharmaceutically acceptable salts. The term “pharmaceutically acceptable salt” refers to conventional acid-addition salts that retain the biological effectiveness and properties of the compounds of formula I and are formed from suitable non-toxic organic or inorganic acids. Sample acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, naphthalenesulfonic acid, naphthalenedisulfonic acid, methanesulfonic acid, ethanesulfonic acid and the like. The chemical modification of a pharmaceutical compound (i.e. a drug) into a salt is a technique well known to pharmaceutical chemists to obtain improved physical and chemical stability, hygroscopicity, flowability and solubility of compounds. See, e.g. Bastin, R. J., et al, Organic Proc. Res. Dev. 4 (2000) 427-435.

The compounds of formula I can contain one or several chiral centers and can then be present in a racemic or in an optically active form. The racemates can be separated according to known methods into the enantiomers. For instance, diastereomeric salts which can be separated by crystallization are formed from the racemic mixtures by reaction with an optically active acid such as e.g. D- or L-camphorsulfonic acid. Alternatively separation of the enantiomers can also be achieved by using chromatography on chiral HPLC-phases (HPLC: High Performance Liquid Chromatography) which are commercially available.

The cycloalkyl ring which is formed by R² and R³ together with the carbon atom to which they are attached is preferably a cyclopentyl or cyclohexyl ring, more preferably a cyclopentyl ring.

The cycloalkyl ring in the definition of the substituents of ring A is preferably a cyclopropyl, cyclobutyl or cyclopentyl ring, more preferably a cyclopropyl ring.

In a particular preferred embodiment, the alkyl, alkenyl or alkynyl encompassed by the R¹ group in formula I which are “optionally substituted one or more times with nitro, cyano or —Y—R⁴” are optionally substituted one to three times, more preferably one to two times, and more preferably one time by nitro, cyano or —Y—R⁴.

In a particular preferred embodiment, the optionally substituted alkyl encompassed by the R⁴ group in formula I is optionally substituted one to six times and more preferably one to three times by halogen, preferably by fluorine or chlorine, more preferably by fluorine; or said optionally substituted alkyl encompassed by the R⁴ group in formula I is preferably substituted one to three times, more preferably one to two times by hydroxy, alkoxy, alkoxyalkoxy, amino, alkylamino, dialkylamino, —C(O)OH or —C(O)NH₂. Examples of such optionally substituted alkyl groups are difluoromethyl, trifluoromethyl, 2,2,2-trifluoroethyl, perfluorethyl, difluoromethoxy, trifluoromethoxy, 2,2,2-trifluoroethoxy, perfluoroethoxy, 2-hydroxy-butyl, 2-hydroxy-ethyl, 2-hydroxy-propyl, 3-hydroxy-butyl, 2,3-dihydroxy-propyl, 2,3-dihydroxy-butyl, 1,2,3-trihydroxy-propyl, 2-hydroxy-pentyl, 2-methoxy-ethyl, 2-ethoxy-ethyl, 4-methoxy-butyl, 2-methoxy-butyl, 2-ethoxy-propyl, 3-propoxy-butyl, 2,3-dimethoxy-propyl, 2-ethoxy-3-methoxy-propyl, 2,3-diethoxy-butyl, 1,2,3-trimethoxy-propyl, 2-methoxy-pentyl, 2-(2-methoxy-ethoxy)-ethyl, 2-(2-ethoxy-ethoxy)-ethyl, 2-(2-propoxy-ethoxy)-ethyl, 3-(2-methoxy-ethoxy)-propyl, 3-(1-methoxy-ethoxy)-propyl, 4-(2-ethoxy-ethoxy)-butyl, 2-amino-butyl, 2-amino-ethyl, 2-amino-propyl, 3-amino-propyl, 3-amino-butyl, 2,3-diamino-propyl, 2-methylamino-butyl, 2-ethylamino-ethyl, 2-dimethylamino-ethyl, 2-dimethylamino-propyl, 3-diethylamino-propyl, 3-amino-butyl, 2,3-diamino-propyl, preferably 2,3-dihydroxy-propyl, 2-methoxy-ethyl, 2-(2-methoxy-ethoxy)-ethyl, trifluoromethyl, and trifluoromethoxy.

In a particular preferred embodiment, the optionally substituted aryl encompassed by the R⁴ group in formula I is optionally substituted one to five times, more preferably one to three times, and more preferably one to two times.

In a particular preferred embodiment, the optionally substituted heteroaryl encompassed by the R⁴ group in formula I is optionally substituted one to two times and more preferably one time.

In a particular preferred embodiment, the optionally substituted ring A in formula I is preferably optionally substituted one to three times, and more preferably one to two times.

Y in formula I is preferably a single bond.

R⁴ in formula I is preferably heterocyclyl, more preferably morpholino.

R² in formula I is preferably hydrogen or alkyl, more preferably alkyl.

R³ in formula I is preferably hydrogen or alkyl, more preferably alkyl.

Preferably R² and R³ in formula I are both alkyl.

X in formula I is preferably a single bond.

In a particular preferred embodiment, Ring A in formula I is preferably a 5 to 6 membered saturated ring. In another preferred emobidment, ring A contains one heteroatom selected from oxygen, nitrogen or sulfur with the remaining ring atoms being carbon atoms. In another preferred embodiment, if ring A contains two heteroatoms such heteroatoms are not adjacent. In another preferred embodiment ring A is optionally substituted one to three times, and more preferably one to two times by alkyl. Examples of such a ring include cyclopentyl, cyclohexyl, cycloheptyl, methyl-cyclopentyl, methyl-cyclohexyl, 1,1-dimethyl-cyclohexyl, 1,3-dimethyl-cyclohexyl, 1,4-dimethyl-cyclohexyl, cycloheptyl, tetrahydrofuran, pyrrolidine, N-methyl-pyrrolidine, N-ethyl-pyrrolidine, N-isopropyl-pyrrolidine, tetrahydrothiophene, [1,3]dioxolane, tetrahydropyran, piperidine, N-methyl-piperidine, N-ethyl-piperidine, N-isopropyl-piperidine, tetrahydrothiopyran, azepane, [1,3]dioxane, [1,3]dioxepane and [1,4]dithiepane; preferably cyclopentyl, cyclohexyl, pyrrolidine, tetrahydrothiophene, tetrahydropyran, piperidine and tetrahydrothiopyran.

In a particular preferred embodiment, if ring A in formula I contains one nitrogen, said nitrogen is optionally substituted once by a substituent selected from the group consisting of:
(1) —CH₂-phenyl;
(2) —C(O)-cycloalkyl (preferably —C(O)-cyclopropyl);
(3) —C(O)-heterocyclyl (preferably —C(O)-morpholino);
(4) —C(O)—(CH₂)ₙ-phenyl, wherein the phenyl is optionally substituted once or twice with halogen (preferably fluorine);
(5) —C(O)—(CH₂)ₙ-heteroaryl (preferably —C(O)—(CH₂)ₙ-thienyl);
(6) —C(O)—NH-phenyl, wherein the phenyl is optionally substituted once or twice with alkyl; and
(7) —S(O)₂-phenyl, wherein the phenyl is optionally substituted once or twice with halogen (preferably fluorine).

Examples of such a substituted nitrogen-containing ring include N-benzyl-pyrrolidine, N-acetyl-pyrrolidine, N-(4-fluoro-benzenesulfonyl)-pyrrolidine, N-(morpholine-4-carbonyl)-piperidine, N-[(2,6-diethyl-phenyl)-aminocarbonyl]-piperidine, N-(2-thiophen-2-yl-acetyl)-piperidine and the like.

n in formula I is preferably 0 or 1.

The ring system formed by fusion of ring A with the pyrazole is an 8 to 10, preferably 8 to 9, membered bicyclic ring system (i.e., 2,4,5,6-tetrahydro-cyclopentapyrazole or the tautomeric form 2,4,5,6-tetrahydro-cyclopentapyrazole; 4,5,6,7-tetrahydro-2H-indazole or the tautomeric form 4,5,6,7-tetrahydro-1H-indazole; 2,4,5,6,7,8-hexahydro-cycloheptapyrazole or the tautomeric form 1,4,5,6,7,8-hexahydro-cycloheptapyrazole) wherein one or two carbon atoms, preferably one carbon atom of ring A (excluding the bridge atoms) can be optionally replaced by a heteroatom independently selected from the group consisting of oxygen, nitrogen and sulfur. If two carbon atoms of ring A are replaced by a heteroatom independently selected from the group consisting of oxygen, nitrogen and sulfur; said heteroatoms are not adjacent.

Examples of such ring systems formed by fusion of ring A with the pyrazole include 2,4,5,6-tetrahydro-cyclopentapyrazole, 4,5,6,7-tetrahydro-2H-indazole, 2,4,5,6,7,8-hexahydro-cycloheptapyrazole, 2,6-dihydro-4H-furo[3,4-c]pyrazole, 2,4,5,6-tetrahydro-pyrrolo[3,4-c]pyrazole, 2,4,5,6-tetrahydro-pyrrolo[2,3-c]pyrazole, 2,6-dihydro-4H-thieno[3,4-c]pyrazole, 2H-[1,3]dioxolo[4,5-c]pyrazole, 2,4,6,7-tetrahydro-pyrano[4,3-c]pyrazole, 2,4,5,7-tetrahydro-pyrano[3,4-c]pyrazole, 4,5,6,7-tetrahydro-1H-pyrazolo[4,3-c]pyridine, 2,4,6,7-tetrahydro-1H-pyrazolo[4,3-c]pyridine, 2,4,6,7-tetrahydro-thiopyrano[4,3-c]pyrazole, 2,4,5,7-tetrahydro-thiopyrano[3,4-c]pyrazole, 2,4-dihydro-[1,3]dioxino[4,5-c]pyrazole, 2,7-dihydro-4,6-dioxa-1,2-diaza-indene, 4,5-dihydro-2H-6,8-dioxa-1,2-diaza-azulene and 6,7-dihydro-2H,5H-4,8-dithia-1,2-diaza-azulene; preferably 2,4,5,6-tetrahydro-cyclopentapyrazole, 4,5,6,7-tetrahydro-2H-indazole, 2,4,5,6-tetrahydro-pyrrolo[3,4-c]pyrazole, 2,6-dihydro-4H-thieno[3,4-c]pyrazole, 2,4,6,7-tetrahydro-pyrano[4,3-c]pyrazole, 4,5,6,7-tetrahydro-1H-pyrazolo[4,3-c]pyridine and 2,4,6,7-tetrahydro-thiopyrano[4,3-c]pyrazole.

In a particular preferred embodiment, if Y is —NH— or —N(alkyl)-, and R⁴ is alkyl, then said alkyl is a substituted once and the substituents are selected from the group consisting of halogen, hydroxy, alkoxy, alkoxyalkoxy, amino, alkylamino, dialkylamino, —C(O)OH and —C(O)NH₂.

In a particular preferred embodiment, if Y is —O—, and R⁴ is alkyl, then said alkyl is substituted once and the substituents are selected from the group consisting of halogen, hydroxy, alkoxyalkoxy, amino, alkylamino, dialkylamino, —C(O)OH and —C(O)NH₂.

The compounds of formula I can exist in different tautomeric forms and in variable mixtures thereof. All tautomeric forms of the compounds of formula I and mixtures thereof are an objective of the invention. For example, the imidazole part of the tricyclic ring system of formula I can exist in two tautomeric forms as shown here below:

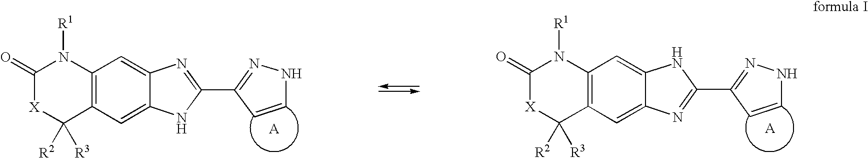

Also, e.g. the pyrazole ring of formula I can form two tautomeric forms as shown here below:

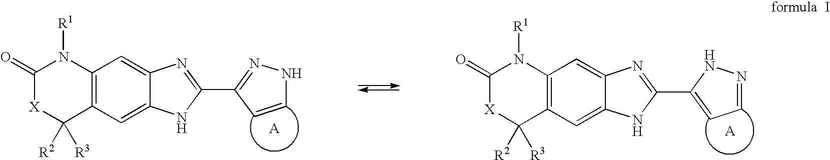

An embodiment of the invention are the compounds according to formula I, wherein ring A is a 5 to 7 membered saturated ring optionally containing one or two heteroatoms independently selected from the group consisting of oxygen, nitrogen and sulfur, with the remaining ring atoms being carbon atoms, wherein said ring A can be optionally substituted one or several times by alkyl.

Another embodiment of the invention are the compounds according to formula I, wherein:
(a) R¹ is hydrogen or alkyl, wherein said alkyl is optionally substituted once by —Y—R⁴;
(b) Y is a single bond;
(c) R⁴ is heterocyclyl, preferably morpholino;
(d) R² is alkyl;
(e) R³ is alkyl;
(f) X is a single bond;
(g) ring A is a 5 to 7, preferably 5 to 6, membered saturated ring optionally containing one or two heteroatoms independently selected from the group consisting of oxygen, nitrogen and sulfur, with the remaining ring atoms being carbon atoms;
wherein said ring A can be optionally substituted one or more times by alkyl, and if said ring A contains one nitrogen, said nitrogen can be optionally substituted once by a substituent selected from the group consisting of:
   (1) —CH₂-phenyl,
   (2) —C(O)-cycloalkyl, preferably —C(O)-cyclopropyl,
   (3) —C(O)-heterocyclyl, preferably —C(O)-morpholino,
   (4) —C(O)—(CH₂)ₙ-phenyl, wherein the phenyl is optionally substituted once or twice with halogen, preferably fluorine,
   (5) —C(O)—(CH₂)ₙ-heteroaryl, preferably —C(O)—(CH₂)ₙ-thienyl,
   (6) —C(O)—NH-phenyl, wherein the phenyl is optionally substituted once or twice with alkyl, and
   (7) —S(O)₂-phenyl, wherein the phenyl is optionally substituted once or twice with halogen, preferably fluorine; and
(h) n is 0 or 1.

Another embodiment of the invention are the compounds according to formula I, wherein:
(a) R¹, R² and R³ are alkyl;
(b) X is a single bond;
(c) ring A is a 5 to 7, preferably 5 to 6, membered saturated ring optionally containing one heteroatom independently selected from the group consisting of oxygen, nitrogen and sulfur, with the remaining ring atoms being carbon atoms;
wherein said ring A can be optionally substituted one or mroel times by alkyl, and if said ring A contains one nitrogen, said nitrogen can be optionally substituted once by a substituent selected from the group consisting of:
   (1) —CH₂-phenyl,
   (2) —C(O)-cycloalkyl,
   (3) —C(O)-heterocyclyl,
   (4) —C(O)—(CH₂)ₙ-phenyl, wherein the phenyl is optionally substituted once or twice with halogen,
   (5) —C(O)—(CH₂)ₙ-thienyl,
   (6) —C(O)—NH-phenyl, wherein the phenyl is optionally substituted once or twice with alkyl, and
   (7) —S(O)₂-phenyl, wherein the phenyl is optionally substituted once or twice with halogen; and
(d) n is 0 or 1.

Another embodiment of the invention are the compounds according to formula I, wherein

ring A is a 5 to 7 membered saturated ring optionally containing one heteroatom independently selected from the group consisting of oxygen, nitrogen and sulfur, with the remaining ring atoms being carbon atoms; wherein said ring A can be optionally substituted one or more times by alkyl.

Another embodiment of the invention are the compounds according to formula I, wherein:
(a) R¹, R² and R³ are alkyl;
(b) X is a single bond; and
(c) ring A is a 5 to 7 membered saturated ring optionally containing one heteroatom independently selected from the group consisting of oxygen, nitrogen and sulfur, with the remaining ring atoms being carbon atoms; wherein said ring A can be optionally substituted one or more times by alkyl.

Another embodiment of the invention are the compounds according to formula I, wherein R⁴ is heterocyclyl.

Another embodiment of the invention are the compounds according to formula I, wherein R⁴ is heterocyclyl; and ring A is a 5 to 7 membered saturated ring optionally containing one heteroatom independently selected from the group consisting of oxygen, nitrogen and sulfur, with the remaining ring atoms being carbon atoms; wherein said ring A can be optionally substituted one or more times by alkyl.

Another embodiment of the invention are the compounds according to formula I, wherein X is a single bond.

Another embodiment of the invention are the compounds according to formula I, wherein X is a single bond; and ring A is a 5 to 7 membered saturated ring optionally containing one heteroatom independently selected from the group consisting of oxygen, nitrogen and sulfur, with the remaining ring atoms being carbon atoms; wherein said ring A can be optionally substituted one or several times by alkyl.

Another embodiment of the invention are the compounds according to formula I, wherein ring A is a 5 to 7 membered saturated hydrocarbon ring.

Another embodiment of the invention are the compounds according to formula I, wherein X is a single bond; and ring A is a 5 to 7, preferably 5 to 6, membered saturated hydrocarbon ring.

Another embodiment of the invention are the compounds according to formula I, wherein:
(a) R¹ is hydrogen or alkyl, wherein said alkyl is optionally substituted once by —Y—R⁴;
(b) Y is a single bond;
(c) R⁴ is heterocyclyl, preferably morpholino;
(d) R² is alkyl;
(e) R³ is alkyl;
(f) X is a single bond; and
(g) ring A is a 5 to 7, preferably 5 to 6, membered saturated hydrocarbon ring.

Such compounds, for example, may be selected from the group consisting of:
7,7-Dimethyl-2-(1,4,5,6-tetrahydro-cyclopentapyrazol-3-yl)-5,7-dihydro-3H-imidazo[4,5-f]indol-6-one;
7,7-Dimethyl-2-(4,5,6,7-tetrahydro-1H-indazol-3-yl)-5,7-dihydro-3H-imidazo[4,5-f]indol-6-one;
5,7,7-Trimethyl-2-(1,4,5,6-tetrahydro-cyclopentapyrazol-3-yl)-5,7-dihydro-3H-imidazo[4,5-f]indol-6-one;
5-Ethyl-7,7-dimethyl-2-(1,4,5,6-tetrahydro-cyclopentapyrazol-3-yl)-5,7-dihydro-3H-imidazo[4,5-f]indol-6-one;
7,7-Dimethyl-5-propyl-2-(1,4,5,6-tetrahydro-cyclopentapyrazol-3-yl)-5,7-dihydro-3H-imidazo[4,5-f]indol-6-one;
5-Isopropyl-7,7-dimethyl-2-(1,4,5,6-tetrahydro-cyclopentapyrazol-3-yl)-5,7-dihydro-3H-imidazo[4,5-f]indol-6-one;
7,7-Dimethyl-5-(3-morpholin-4-yl-propyl)-2-(1,4,5,6-tetrahydro-cyclopentapyrazol-3-yl)-5,7-dihydro-3H-imidazo[4,5-f]indol-6-one;
5-Ethyl-7,7-dimethyl-2-(4,5,6,7-tetrahydro-1H-indazol-3-yl)-5,7-dihydro-3H-imidazo[4,5-f]indol-6-one;
5-Isopropyl-7,7-dimethyl-2-(4,5,6,7-tetrahydro-1H-indazol-3-yl)-5,7-dihydro-3H-imidazo[4,5-f]indol-6-one;
7,7-Dimethyl-5-(3-morpholin-4-yl-propyl)-2-(4,5,6,7-tetrahydro-1H-indazol-3-yl)-5,7-dihydro-3H-imidazo[4,5-f]indol-6-one; and
5,7,7-Triethyl-2-(4,5,6,7-tetrahydro-1H-indazol-3-yl)-5,7-dihydro-3H-imidazo[4,5-f]indol-6-one.

Another embodiment of the invention are the compounds according to formula I, wherein:
(a) R¹, R² and R³ are alkyl;
(b) X is a single bond;
(c) ring A is a 5 to 7, preferably 5 to 6, membered saturated ring containing one heteroatom independently selected from the group consisting of oxygen, nitrogen and sulfur, with the remaining ring atoms being carbon atoms; wherein said ring A can be optionally substituted one or more times by alkyl; wherein if said ring A contains one nitrogen, said nitrogen can be optionally substituted once by a substituent selected from the group consisting of:
   (1) —CH₂-phenyl,
   (2) —C(O)-cycloalkyl, preferably —C(O)-cyclopropyl,
   (3) —C(O)-heterocyclyl, preferably —C(O)-morpholino,
   (4) —C(O)—(CH₂)ₙ-phenyl, wherein the phenyl is optionally substituted once or twice with halogen, preferably fluorine,
   (5) —C(O)—(CH₂)ₙ-thienyl,
   (6) —C(O)—NH-phenyl, wherein the phenyl is optionally substituted once or twice with alkyl, and
   (7) —S(O)₂-phenyl, wherein the phenyl is optionally substituted once or twice with halogen, preferably fluorine; and
(d) n is 0 or 1.

Such compounds, for example, may be selected from the group consisting of:
5-Ethyl-7,7-dimethyl-2-(1,4,6,7-tetrahydro-pyrano[4,3-c]pyrazol-3-yl)-5,7-dihydro-3H-imidazo[4,5-f]indol-6-one;
2-(5-Benzyl-4,5,6,7-tetrahydro-1H-pyrazolo[4,3-c]pyridin-3-yl)-5-ethyl-7,7-dimethyl-5,7-dihydro-3H-imidazo[4,5-f]indol-6-one;
5-Ethyl-7,7-dimethyl-2-(4,5,6,7-tetrahydro-1H-pyrazolo[4,3-c]pyridin-3-yl)-5,7-dihydro-3H-imidazo[4,5-f]indol-6-one;
2-(5-Benzyl-1,4,5,6-tetrahydro-pyrrolo[3,4-c]pyrazol-3-yl)-5-ethyl-7,7-dimethyl-5,7-dihydro-3H-imidazo[4,5-f]indol-6-one;
5-Ethyl-7,7-dimethyl-2-(1,4,6,7-tetrahydro-thiopyrano[4,3-c]pyrazol-3-yl)-5,7-dihydro-3H-imidazo[4,5-f]indol-6-one;
2-(5-Cyclopropanecarbonyl-1,4,5,6-tetrahydro-pyrrolo[3,4-c]pyrazol-3-yl)-5-ethyl-7,7-dimethyl-5,7-dihydro-3H-imidazo[4,5-f]indol-6-one;
5-Ethyl-2-{5-[2-(4-fluoro-phenyl)-acetyl]-4,5,6,7-tetrahydro-1H-pyrazolo[4,3-c]pyridin-3-yl}-7,7-dimethyl-5,7-dihydro-3H-imidazo[4,5-f]indol-6-one;
5-Ethyl-2-[5-(4-fluoro-benzenesulfonyl)-4,5,6,7-tetrahydro-1H-pyrazolo[4,3-c]pyridin-3-yl]-7,7-dimethyl-5,7-dihydro-3H-imidazo[4,5-f]indol-6-one;
5-Ethyl-7,7-dimethyl-2-[5-(morpholine-4-carbonyl)-4,5,6,7-tetrahydro-1H-pyrazolo[4,3-c]pyridin-3-yl]-5,7-dihydro-3H-imidazo[4,5-f]indol-6-one;
5-Ethyl-2-[5-(4-fluoro-benzoyl)-4,5,6,7-tetrahydro-1H-pyrazolo[4,3-c]pyridin-3-yl]-7,7-dimethyl-5,7-dihydro-3H-imidazo[4,5-f]indol-6-one;
2-(4,6-Dihydro-1H-thieno[3,4-c]pyrazol-3-yl)-5-ethyl-7,7-dimethyl-5,7-dihydro-3H-imidazo[4,5-f]indol-6-one;
5-Ethyl-7,7-dimethyl-2-[5-(2-thiophen-2-yl-acetyl)-1,4,5,6-tetrahydro-pyrrolo[3,4-c]pyrazol-3-yl]-5,7-dihydro-3H-imidazo[4,5-f]indol-6-one;
2-(4,6-Dihydro-1H-thieno[3,4-c]pyrazol-3-yl)-5-isopropyl-7,7-dimethyl-5,7-dihydro-3H-imidazo[4,5-f]indol-6-one;
3-(5-Ethyl-7,7-dimethyl-6-oxo-3,5,6,7-tetrahydro-imidazo[4,5-f]indol-2-yl)-4,6-dihydro-1H-pyrrolo[3,4-c]pyrazole-5-carboxylic acid (2,6-diethyl-phenyl)-amide;
5-Ethyl-2-[5-(4-fluoro-benzenesulfonyl)-1,4,5,6-tetrahydro-pyrrolo[3,4-c]pyrazol-3-yl]-7,7-dimethyl-5,7-dihydro-3H-imidazo[4,5-f]indol-6-one;
3-(5-Isopropyl-7,7-dimethyl-6-oxo-3,5,6,7-tetrahydro-imidazo[4,5-f]indol-2-yl)-4,6-dihydro-1H-pyrrolo[3,4-c]pyrazole-5-carboxylic acid (2,6-diethyl-phenyl)-amide; and
5-Isopropyl-7,7-dimethyl-2-[5-(2-thiophen-2-yl-acetyl)-1,4,5,6-tetrahydro-pyrrolo[3,4-c]pyrazol-3-yl]-5,7-dihydro-3H-imidazo[4,5-f]indol-6-one.

Another embodiment of the invention are the compounds according to formula I, wherein:
R¹, R² and R³ are alkyl;
X is a single bond; and
ring A is a 5 to 7, preferably 5 to 6, membered saturated ring containing one heteroatom independently selected from the group consisting of oxygen, nitrogen and sulfur, with the remaining ring atoms being carbon atoms.

Another embodiment of the invention are the compounds according to formula I, wherein:
(a) R¹, R² and R³ are alkyl;
(b) X is a single bond;
(c) ring A is a 5 to 7, preferably 5 to 6, membered saturated ring containing one nitrogen and the remaining ring atoms being carbon atoms, wherein said nitrogen is substituted once by a substituent selected from the group consisting of:
   (1) —CH₂-phenyl,
   (2) —C(O)-alkyl,
   (3) —C(O)-cycloalkyl,
   (4) —C(O)-heterocyclyl,
   (5) —C(O)—(CH₂)ₙ-phenyl, wherein the phenyl is optionally substituted once or twice with halogen, alkyl, alkoxy, nitro, amino, alkylamino, dialkylamino, cyano, trifluoromethyl or trifluoromethoxy,
   (6) —C(O)—(CH₂)ₙ-heteroaryl,
   (7) —C(O)—NH-phenyl, wherein the phenyl is optionally substituted once or twice with halogen, alkyl, alkoxy, nitro, amino, alkylamino, dialkylamino, cyano, trifluoromethyl or trifluoromethoxy, and
   (8) —S(O)₂-phenyl, wherein the phenyl is optionally substituted once or twice with halogen, alkyl, alkoxy, nitro, amino, alkylamino, dialkylamino, cyano, trifluoromethyl or trifluoromethoxy; and
(d) n is 0, 1 or 2.

Another embodiment of the invention are the compounds according to formula I, wherein:
(a) R¹, R² and R³ are alkyl;
(b) X is a single bond;
(c) ring A is a 5 to 7, preferably 5 to 6, membered saturated ring containing one nitrogen and the remaining ring atoms being carbon atoms, wherein said nitrogen is substituted once by a susbstituent selected from the group consisting of:
   (1) —CH₂-phenyl,
   (2) —C(O)-cycloalkyl, preferably —C(O)-cyclopropyl,
   (3) —C(O)-heterocyclyl, preferably —C(O)-morpholino,
   (4) —C(O)—(CH₂)ₙ-phenyl, wherein the phenyl is optionally substituted once or twice with halogen, preferably fluorine,
   (5) —C(O)—(CH₂)ₙ-thienyl,
   (6) —C(O)—NH-phenyl, wherein the phenyl is optionally substituted once or twice with alkyl, and
   (7) —S(O)₂-phenyl, wherein the phenyl is optionally substituted once or twice with halogen, preferably fluorine; and
(d) n is 0 or 1.

Another embodiment of the invention is a process for the preparation of the compounds of formula I comprising the steps of:
(a) reacting a compound of formula II: 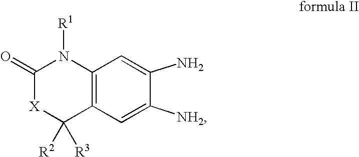
wherein X, R¹, R² and R³ have the significance given above for formula I, with a compound of formula III: 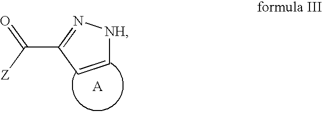
wherein ring A has the significance given above for formula I and Z is —OH, —Cl, —H, —OMe or hydroxybenzotriazole,
to obtain the compounds of formula I: 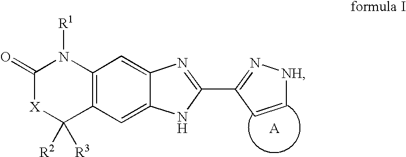
   (b) isolating the compounds of formula I; and
   (c) optionally converting the compounds of formula I into their pharmaceutically acceptable salts or esters.

The tricyclic compounds of formula I, or a pharmaceutically acceptable salt or ester thereof, which are the subject of the present invention, may be prepared by any process known to be applicable to the preparation of chemically-related compounds. Such processes, when used to prepare a compound of the formula I, or a pharmaceutically-acceptable salt or ester thereof, are illustrated by the following representative schemes 1 to 3 and examples in which, unless otherwise stated, R¹, R², R³, R⁴, X, Y and ring A have the definition previously given for formula I. Necessary starting materials are either commercially available or they may be obtained by standard procedures of organic chemistry. The preparation of such starting materials is described within the accompanying examples or in the literature cited below with respect to scheme 1 to 3. Alternatively necessary starting materials are obtainable by analogous procedures to those illustrated which are within the ordinary skill of an organic chemist.

The tricyclic fused imidazole ring of formula I can be formed by different synthetic pathways in analogy to methods described in the literature (Mertens, A., et al., J. Med. Chem. 30 (1987) 1279-1287; U.S. Pat. No. 4,695,567A).

One route for the preparation of compounds of formula I (Scheme 1) starts from diamines of formula II which can be reacted with carboxylic acids (compounds of formula III wherein Z is —OH), acid chlorides (compounds of formula III wherein Z is —Cl), aldehydes (compounds of formula III wherein Z is —H), methyl carboxylates (compounds of formula III wherein Z is —OMe) or activated esters (compounds of formula III wherein A is e.g. hydroxybenzotriazole). For detailed procedures see Mertens, A., et al., J. Med. Chem. 30 (1987) 1279-1287 and U.S. Pat. No. 4,695,567A.

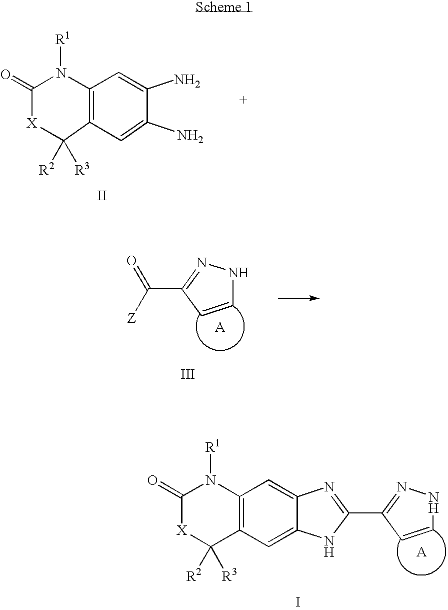

In scheme 1, A, R¹, R², R³ and X have the significance given previously for formula I and Z is —OH, —Cl, —H, —OMe or e.g. hydroxybenzotriazole.

The synthesis of diamines of formula II or precursors thereof is described in Mertens, A., et al., J. Med. Chem. 30 (1987) 1279-1287; von der Saal, W., et al., J. Med. Chem. 32 (1989) 1481-1491; U.S. Pat. No. 4,666,923A, U.S. Pat. No. 4,695,567A, U.S. Pat. No. 4,863,945A and U.S. Pat. No. 4,985,448A. For instance, the diamines of formula II, wherein A is a single bond are named IIa and can be synthesized according to U.S. Pat. No. 4,666,923A, DE 34 10 168 and Mertens, A., et al., J. Med. Chem. 30 (1987) 1279-1287 as shown in Scheme 2a:

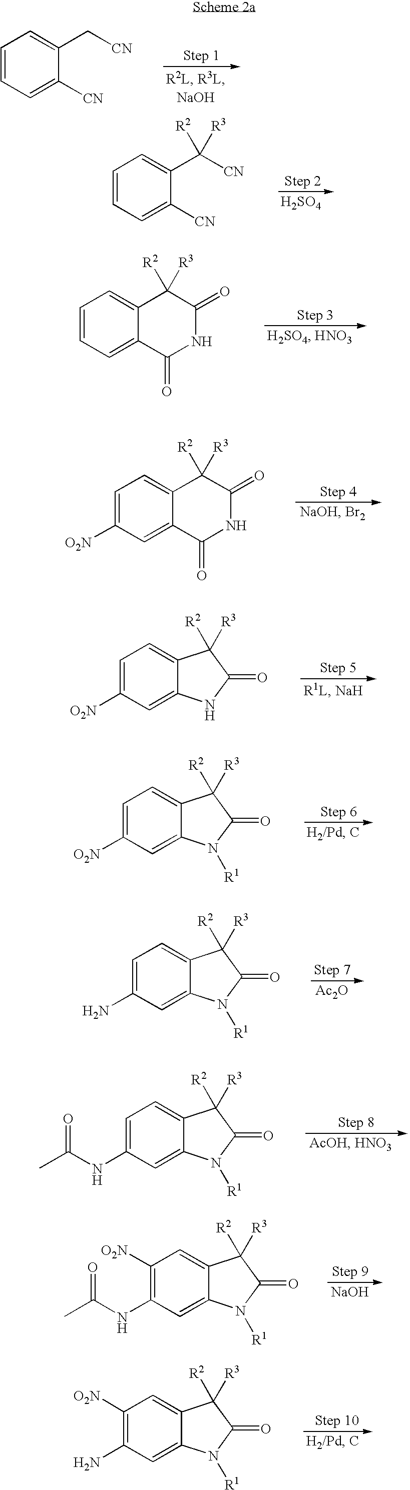

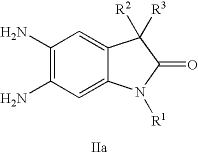

In scheme 2a, R¹, R² and R³ have the significance given previously for formula I and L represents a leaving group such as iodine, bromine, chlorine, triflate and the like.

In an alternative procedure diamines of formula IIa can be obtained by an alkylation of diamines of formula IIb (compounds II wherein A is a single bond and R¹ is hydrogen) as shown in scheme 2b.

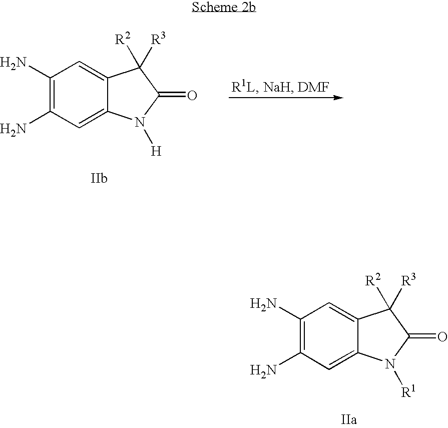

Diamines of formula IIb can be synthesized according to scheme 1 under omission of step 5.

Annelated pyrazoles of formula III in scheme 1 are either commercially available or they can be prepared by different synthetic routes according to the nature of “Z” and “ring A”. If “Z” is hydroxy the corresponding annelated pyrazole 3-carboxylic acids are named IIIa and can be manufactured e.g. as shown in the following scheme 3.

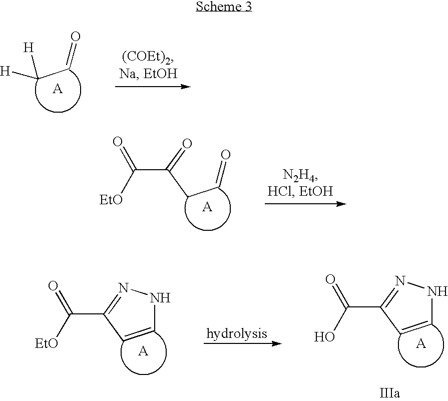

In scheme 3, A has the significance as given previously for formula I. Various cyclic carbonyl compounds with an α-methylene group undergo mixed Claisen condensation with diethyl oxalate. The resulting α,γ-diketo esters can be condensed with hydrazine to give the annelated pyrazole. After hydrolysis of the ester functionality of the desired annelated pyrazole 3-carboxylic acids IIIa are obtained (see e.g. van Herk, T., et al., J. Med. Chem. 46 (2003) 3945-3951).

The preparation of compounds of formula III wherein Z is —OEt and A is a 5 to 7 membered saturated ring containing one nitrogen atom wherein the nitrogen is substituted by methyl (especially 4-methyl-1,4,5,6-tetrahydro-pyrrolo[3,2-c]pyrazole-3-carboxylic acid ethyl ester, 4-methyl-4,5,6,7-tetrahydro-1H-pyrazolo[4,3-b]pyridine-3-carboxylic acid ethyl ester and 4-methyl-1,4,5,6,7,8-hexahydro-1,2,4-triaza-azulene-3-carboxylic acid ethyl ester) are described in Möhrle, H., et al., Chem. Ber. 119 (1986) 3591-3599, starting from the corresponding lactame acetals by reaction with diazo-acetic acid ester.

Certain substituents on the groups encompassed by R¹ may not be inert to the conditions of the synthesis sequences described above and may require protection by standard protecting groups known in the art. For instance, an amino or hydroxyl group may be protected as an acetyl or tert.-butoxycarbonyl derivative. Alternatively, some substituents may be derived from others at the end of the reaction sequence. For instance, a compound of formula I may be synthesized bearing a nitro-, an ethoxycarbonyl, a sulfonic acid substituent on the group R¹, which substituents are finally converted to an amino-, alkylamino-, dialkylamino-, acylamino-, alkylsulfonylamino, arylsulfonylamino substituent, or to a carboxamide substituent, or to a sulfonamide substituent by standard procedures.

Pharmaceutical compositions containing a compound of the present invention or a pharmaceutically acceptable salt or ester thereof and a therapeutically acceptable carrier are an object of the present invention, as is a process for their production, which comprises bringing one or more compounds of the present invention and/or pharmaceutically acceptable salts or esters and, if desired, one or more other therapeutic substances into a galenical administration form together with one or more pharmaceutically acceptable carriers.

In accordance with the invention the compounds of the present invention as well as their pharmaceutically acceptable salts or esters are useful in the control or prevention of illnesses. Based on their Aurora tyrosine kinase inhibition and their antiproliferative activity, said compounds are useful for the treatment of diseases such as cancer in humans or animals and for the production of corresponding pharmaceutical compositions. The dosage depends on various factors such as manner of administration, species, age and/or individual state of health.

An embodiment of the invention is a pharmaceutical composition, containing one or more compounds according to formula I, together with pharmaceutically acceptable excipients.

Another embodiment of the invention is a pharmaceutical composition containing one or more compounds of formula I as active ingredients together with pharmaceutically acceptable adjuvants for the treatment of diseases mediated by an inappropriate activation of Aurora family tyrosine kinases.

Another embodiment of the invention is a pharmaceutical composition, containing one or more compounds according to formula I, for the inhibition of tumor growth.

Another embodiment of the invention is a pharmaceutical composition containing one or more compounds of formula I as active ingredients together with pharmaceutically acceptable adjuvants for the treatment of colorectal, breast, lung, prostate, pancreatic, gastric, bladder, ovarian, melanoma, neuroblastoma, cervical, kidney or renal cancers, leukemias or lymphomas.

Another embodiment of the invention is a pharmaceutical composition containing one or more compounds of formula I as active ingredients together with pharmaceutically acceptable adjuvants for the treatment of acute-myelogenous leukemia (AML, acute lymphocytic leukemia (ALL) and gastrointestinal stromal tumor (GIST).

Another embodiment of the invention is the use of one or more compounds of formula I for the manufacture of pharmaceutical compositions for the treatment of diseases mediated by an inappropriate activation of Aurora family tyrosine kinases.

Another embodiment of the invention is the use of a compound according to formula I, for the manufacture of corresponding pharmaceutical compositions for the inhibition of tumor growth.

Another embodiment of the invention is the use of a compound according to formula I, for the manufacture of corresponding pharmaceutical compositions for the treatment of colorectal, breast, lung, prostate, pancreatic, gastric, bladder, ovarian, melanoma, neuroblastoma, cervical, kidney or renal cancers, leukemias or lymphomas.

Another embodiment of the invention is the use of a compound according to formula I, for the manufacture of pharmaceutical compositions for the treatment of acute-myelogenous leukemia (AML, acute lymphocytic leukemia (ALL) and gastrointestinal stromal tumor (GIST).

Another embodiment of the invention is the use of the compounds of formula I as Aurora A tyrosine kinase inhibitors.

Another embodiment of the invention is the use of the compounds of formula I as anti-proliferating agents.

Another embodiment of the invention is the use of one or more compounds of formula I for the treatment of cancer.

Pharmacological Activity

The compounds of formula I and their pharmaceutically acceptable salts or esters possess valuable pharmacological properties. It has been found that said compounds show activity as inhibitors of the Aurora kinase family and also show anti-proliferative activity. Consequently the compounds of the present invention are useful in the therapy and/or prevention of illnesses with known over-expression of kinases of the Aurora family preferably Aurora A, especially in the therapy and/or prevention of illnesses mentioned above. The activity of the present compounds as inhibitors of the Aurora kinase family is demonstrated by the following biological assay:

IC₅₀ Determination for Inhibitors of Aurora A

Assay Principle

Aurora A is a serine threonine kinase involved in spindle assembly and chromosome segregation.

The assay is a typically ELISA-type assay where substrate (GST-Histone H3) is coupled to the assay-plate and is phosphorylated by the kinase. Phosphorylation is detected by a mouse anti-Phosphopeptid mAb and an HRP-labeled anti-mouse pAb. The assay is validated for IC₅₀-determination.

Kinase activities were measured by Enzyme-Linked Immunosorbent Assay (ELISA): Maxisorp 384-well plates (Nunc) were coated with recombinant fusion protein comprising residues 1-15 of HistoneH3 fused to the N-terminus of Glutathione-S-Transferase. Plates were then blocked with a solution of 1 mg/mL I-block (a highly purified form of casein, Tropix cat. no. T2015) in phosphate-buffered saline. Kinase reactions were carried out in the wells of the ELISA plate by combining an appropriate amount of mutant Aurora A kinase with test compound and 30 μM ATP. The reaction buffer was 10× Kinase Buffer (Cell Signaling cat. no. 9802) supplemented with 1 μg/mL I-block. Reactions were stopped after 40 minutes by addition of 25 mM EDTA. After washing, substrate phosphorylation was detected by addition of anti-phospho-Histone H3 (Ser 10) 6G3 mAb (Cell Signaling cat. no. 9706) and sheep anti-mouse pAb-HRP (Amersham cat. no. NA931V), followed by colorimetric development with TMB (3,3′,5,5′-tetramethylbenzidine from Kirkegaard & Perry Laboratories). After readout of the adsorbance, IC₅₀ values were calculated using a non-linear curve fit (XLfit software (ID Business Solution Ltd., Guilford, Surrey, UK)). The results are shown are shown in Table 1.

TABLE 1
Results:

| | IC50 Aurora A kinase |
|---|---|
| Example No. | inhibition [μM] |
| 5 | 0.017 |
| 9 | 0.012 |
| 11 | 0.016 |
| 16 | 0.055 |
| 17 | 0.014 |
| 13, 18, 19, 20, 21, 22, 24, 26, 28 | 0.001-0.500 |

Antiproliferative Activity

The activity of the present compounds as antiproliferative agents is demonstrated by the following biological assay:

Viability Assay in HCT 116 Cells

A viability assay was performed using the CellTiter-Glo® Luminescent Cell Viability Assay (see Promega Corporation's Technical Bulletin No. 288, pp. 1-11 [revised February 2004] which is hereby incorporated by reference in its entirety). This assay is a homogeneous method of determining the number of viable cells in culture based on quantitation of the ATP present, an indicator of metabolically active cells. The assay is designed for use with multiwell formats, making it ideal for automated high-throughput screening (HTS), cell proliferation and cytotoxicity assays. The homogenous assay procedure involves adding a single reagent (containing luciferase, luciferan substrate, and buffer) directly to cells cultured in serum-supplemented medium. Cell washing, removal of medium and multiple pipetting steps are not required. The system detects as few as 15 cells/well in a 384-well format in 10 minutes after adding reagent and mixing.

The homogeneous “add-mix-measure” format results in cell lysis and generation of a luminescent signal proportional to the amount of ATP present. The amount of ATP is directly proportional to the number of cells present in culture. The above-referenced assay generates a “glow-type” luminescent signal, produced by the luciferase reaction, which has a half-life generally greater than five hours, depending on cell type and medium used. The extended half-life eliminates the need to use reagent injectors and provides flexibility for continuous or batch mode processing of multiple plates. The unique homogeneous format avoids errors that may be introduced by other ATP measurement methods that require multiple steps.

HCT 116 cells (human colon carcinoma, ATCC-No. CCl-247) were cultivated in RPMI 1640 medium with GlutaMAX™ I (cell culture media that contains L-Alanyl-L-Glutamine [a stabilized a form/source of L-Glutamine] from Invitrogen, Cat-No. 61870-010), 2.5% Fetal Calf Serum (FCS, Sigma Cat-No. F4135 (FBS)); 100 Units/ml penicillin/100 μg/ml streptomycin (=Pen/Strep from Invitrogen Cat. No. 15140). For the assay the cells were seeded in 384 well plates, 1000 cells per well, in the same medium. The next day the test compounds were added in various concentrations ranging from 30 μM to 0.0015 μM (10 concentrations, 1:3 diluted). After 5 days the viability assay was done according to the instructions of the manufacturer. In brief: the cell-plate was equilibrated to room temperature for approximately 30 minutes and then reagent (containing luciferase, luciferan substrate, and buffer) was added. The contents were carefully mixed for 15 minutes to induce cell lysis. After 45 minutes the luminescent signal was measured in Victor 2, (scanning multiwell spectrophotometer, Wallac).

Details:

1st. Day:

Medium: RPMI 1640 with cell culture media containing L-Alanyl-L-Glutamine [GlutaMAX™ I (Invitrogen, Cat-No. 61870)], 5% FCS (Sigma Cat.-No. F4135), Pen/Strep (Invitrogen, Cat No. 15140).

HCT116 (ATCC-No. CCl-247): 1000 cells in 60 μl per well of 384 well plate (Greiner 781098, μClear-plate white)

After seeding incubate plates 24 h at 37° C., 5% CO₂

2nd. Day: Induction (Treatment with Compounds, 10 Concentrations):

In order to achieve a final concentration of 30 μM as highest concentration 3.5 μl of 10 mM compound stock solution were added directly to 163 μl media. Then step (d) of the dilution procedure described below, was followed.

In order to achieve the second highest to the lowest concentrations, a serial dilution with dilution steps of 1:3 was followed according to the procedure (a-d) as described here below:

(a) for the second highest concentration add 10 μl of 10 mM stock solution of compound to 20 μl dimethylsulfoxide (DMSO)

(b) dilute 8×1:3 (always 10 μl to 20 μl DMSO) in this DMSO dilution row (results in 9 wells with concentrations from 3333.3 μM to 0.51 μM)

(c) dilute each concentration 1: 47.6 (3.5 μl compound dilution to 163 μl media)

(d) add 10 μl of every concentration to 60 μl media in the cell plate

resulting in final concentration of DMSO: 0.3% in every well
and resulting in 10 final concentration of compounds ranging from 30 μM to 0.0015 μM.
Each compound is tested in triplicate.
Incubate 120 h (5 days) at 37° C., 5% CO₂

Analysis:

Add 30 μl of reagent containing luciferase, luciferan substrate, and buffer (lyophilized) per well,

shake 15 minutes at room temperature

incubate further 45 minutes at room temperature without shaking

Measurement:

Victor 2 scanning multiwell spectrophotometer (Wallac), Luminescence mode (0.5 sec/read, 477 nm)

Determine IC50 using a non-linear curve fit (XLfit® software [ID Business Solution Ltd., Guilford, Surrey, UK])

With all compounds a significant inhibition of HCT 116 cell viability was detected, which is exemplified by the compounds shown in Table 2.

TABLE 2
Results:

| | IC50 |
|---|---|
| Example No. | HCT 116 [μM] |
| 2 | 0.430 |
| 4 | 0.224 |
| 6 | 0.682 |
| 9 | 0.108 |
| 15 | 1.504 |
| 24 | 0.741 |
| 28 | 0.338 |
| 1, 3, 7, 8, 11, 13, 16, 17, 18, 21, 23, 25, 26 | 0.100-2.000 |

The compounds according to this invention and their pharmaceutically acceptable salts or esters can be used as medicaments, e.g. in the form of pharmaceutical compositions. The pharmaceutical compositions can be administered orally, e.g. in the form of tablets, coated tables, dragées, hard and soft gelatine capsules, solutions, emulsions or suspensions. The administration can, however, also be effected rectally, e.g. in the form of suppositories, or parenterally, e.g. in the form of injection solutions.

The above-mentioned pharmaceutical compositions can be obtained by processing the compounds according to this invention with pharmaceutically inert, inorganic or organic carriers. For example, lactose, corn starch or derivatives thereof, talc, stearic acids or it's salts and the like can be used as carriers for tablets, coated tablets, dragées and hard gelatine capsules. Suitable carriers for soft gelatine capsules are, for example, vegetable oils, waxes, fats, semi-solid and liquid polyols and the like. However, depending on the nature of the active substance, carriers may not be required for some soft gelatine capsules. Suitable carriers for the production of solutions and syrups are, for example, water, polyols, glycerol, vegetable oil and the like. Suitable carriers for suppositories are, for example, natural or hardened oils, waxes, fats, semi-liquid or liquid polyols and the like.

The pharmaceutical compositions can, moreover, contain preservatives, solubilizers, stabilizers, wetting agents, emulsifiers, sweeteners, colorants, flavorants, salts for varying the osmotic pressure, buffers, masking agents or antioxidants. They can also contain still other therapeutically valuable substances.

A pharmaceutical composition may comprise, for example, the following:

a) Tablet Formulation (Wet Granulation):

| Item | Ingredients | Mg/tablet | | | |
|---|---|---|---|---|---|
| 1. | Compound of formula I | 5 | 25 | 100 | 500 |
| 2. | Lactose Anhydrous DTG (direct | 125 | 105 | 30 | 150 |
| | tabletting grade) | | | | |
| 3. | Sta-Rx 1500 (pre-gelatinized starch | 6 | 6 | 6 | 30 |
| | powder) | | | | |
| 4. | Microcrystalline Cellulose | 30 | 30 | 30 | 150 |
| 5. | Magnesium Stearate | 1 | 1 | 1 | 1 |
| | Total | 167 | 167 | 167 | 831 |

Manufacturing Procedure:
1. Mix items 1, 2, 3 and 4 and granulate with purified water.
2. Dry the granules at 50° C.
3. Pass the granules through suitable milling equipment.
4. Add item 5 and mix for three minutes; compress on a suitable press.

b) Capsule Formulation:

| Item | Ingredients | mg/capsule | | | |
|---|---|---|---|---|---|
| 1. | Compound of formula I | 5 | 25 | 100 | 500 |
| 2. | Hydrous Lactose | 159 | 123 | 148 | — |
| 3. | Corn Starch | 25 | 35 | 40 | 70 |
| 4. | Talc | 10 | 15 | 10 | 25 |
| 5. | Magnesium Stearate | 1 | 2 | 2 | 5 |
| | Total | 200 | 200 | 300 | 600 |

Manufacturing Procedure:
1. Mix items 1, 2 and 3 in a suitable mixer for 30 minutes.
2. Add items 4 and 5 and mix for 3 minutes.
3. Fill into a suitable capsule.
   c) Micro Suspension
1. Weigh 4.0 g glass beads in custom made tube GL 25, 4 cm (the beads fill half of the tube).
2. Add 50 mg compound, disperse with spatulum and vortex.
3. Add 2 ml gelatin solution (weight beads:gelatin solution=2:1) and vortex.
4. Cap and wrap in aluminum foil for light protection.
5. Prepare a counter balance for the mill.
6. Mill for 4 hours, 20/s in a Retsch mill (for some substances up to 24 hours at 30/s).
7. Extract suspension from beads with two layers of filter (100 μm) on a filter holder, coupled to a recipient vial by centrifugation at 400 g for 2 min.
8. Move extract to measuring cylinder.
9. Repeat washing with small volumes (here 1 ml steps) until final volume is reached or extract is clear.
10. Fill up to final volume with gelatin and homogenize.

The following examples and references are provided to aid the understanding of the present invention, the true scope of which is set forth in the appended claims. It is understood that modifications can be made in the procedures set forth without departing from the spirit of the invention.

Experimental Procedures:

A: Starting Materials

### A1. Preparation of 5,6-diamino-1-ethyl-3,3-dimethyl-1,3-dihydro-indol-2-one

### i) 1-Ethyl-3,3-dimethyl-6-nitro-1,3-dihydro-indol-2-one

A solution of 3,3-dimethyl-6-nitro-1,3-dihydro-indol-2-one (6 g, 29.10 mmol) in anhydrous N,N-dimethylformamide (DMF) (35 ml) was treated with sodium hydride. The resulting suspension was stirred for 1 h at 60° C. A solution of bromo-ethane (2.17 mL, 3.17 g, 29.10 mmol) in DMF (10 ml) was added. The mixture was allowed to cool to room temperature and stirred for 1 h. After removal of the solvent the mixture was quenched with water (100 ml) and extracted with ethyl acetate (3×100 ml). The extract was dried over Na₂SO₄, evaporated and the crude product was purified by column chromatography on silica gel. Elution with ethyl acetate/n-heptane (1:3) yielded 5.94 g (87%) of a yellow solid.

MS: M=235.3 (ESI+)

¹H-NMR (400 MHz, DMSO): δ (ppm)=1.16 (t, 3H), 1.32 (s, 6H), 3.81 (q, 2H), 7.66 (d, 1H), 7.86 (s, 1H), 7.97 (d, 1H)

### ii) 6-Amino-1-ethyl-3,3-dimethyl-1,3-dihydro-indol-2-one

To a solution of 1-ethyl-3,3-dimethyl-6-nitro-1,3-dihydro-indol-2-one (5.9 g, 25.19 mmol) in methanol/tetrahydrofuran (THF) (1:1, 80 ml) palladium on charcoal (10%, 1.2 g) was added and the mixture hydrogenated at room temperature for 4 h. After filtration and evaporation of the solvents 5.05 g (98%) 6-amino-1-ethyl-3,3-dimethyl-1,3-dihydro-indol-2-one was isolated as white solid.

MS: M=205.0 (API+)

¹H-NMR (400 MHz, DMSO): δ (ppm)=1.11 (t, 3H), 1.17 (s, 6H), 3.58 (q, 2H), 5.12 (br, 2H), 6.21 (d, 1H), 6.25 (s, 1H), 6.92 (d, 1H)

### iii) N-(1-Ethyl-3,3-dimethyl-2-oxo-2,3-dihydro-1H-indol-6-yl)-acetamide

A solution of 6-amino-1-ethyl-3,3-dimethyl-1,3-dihydro-indol-2-one (5.05 g, 24.72 mmol) in acetic anhydride (80 ml) was stirred at room temperature for 4 h. The mixture was poured onto ice water (150 ml), allowed to warm to room temperature and was stirred again for 2 h. After extraction with ethyl acetate (3×100 ml), the combined organic layers were washed with sat. NaHCO₃-solution (3×100 ml), brine (100 ml) and dried over sodium sulfate. After removal of the solvent the crude product was purified by column chromatography on silica gel (ethyl acetate/n-heptane 1:1) yielding 5.6 g (91%) N-(1-ethyl-3,3-dimethyl-2-oxo-2,3-dihydro-1H-indol-6-yl)-acetamide as light yellow solid.

MS: M=247.1 (API+)

¹H-NMR (400 MHz, DMSO): δ (ppm)=1.13 (t, 3H), 1.23 (s, 6H), 2.04 (s, 3H), 3.63 (q, 2H), 7.12 (d, 1H), 7.23 (d, 1H), 7.37 (s, 1H), 9.97 (br, 1H)

### iv) N-(1-ethyl-3,3-dimethyl-5-nitro-2-oxo-2,3-dihydro-1H-indol-6-yl)-acetamide

To a solution of N-(1-ethyl-3,3-dimethyl-2-oxo-2,3-dihydro-1H-indol-6-yl)-acetamide (5.6 g, 22.73 mmol) in acetic anhydride (70 ml) nitric acid (100%, 1.96 g, 1.29 ml, 31.2 mmol) was added at 0° C. The mixture was stirred for 30 min, then poured onto ice water (150 ml). After stirring for 4 h the mixture was extracted with ethyl acetate (3×100 ml). The combined organic layers were washed with sodium hydroxide solution (1M, 100 ml) and water (100 ml), dried over sodium sulfate and concentrated. The crude product was purified by column chromatography on silica gel (ethyl acetate/n-heptane 1:1) to yield 5.2 g (78%) N-(1-ethyl-3,3-dimethyl-5-nitro-2-oxo-2,3-dihydro-1H-indol-6-yl)-acetamide as a yellow solid.

MS: M=292.0 (API+)

¹H-NMR (400 MHz, DMSO): δ (ppm)=1.16 (t, 3H), 1.31 (s, 6H), 2.13 (s, 3H), 3.71 (m, 2H), 7.54 (s, 1H), 8.12 (s, 1H), 10.39 (br, 1H)

### v) 6-Amino-1-ethyl-3,3-dimethyl-5-nitro-1,3-dihydro-indol-2-one

N-(1-ethyl-3,3-dimethyl-5-nitro-2-oxo-2,3-dihydro-1H-indol-6-yl)-acetamide (5.2 g, 17.85 mmol) was dissolved in ethanol (40 ml). After addition of hydrochloric acid (25%, 8 ml, 81.44 mmol) the mixture was stirred under reflux for 3 h. The reaction mixture was allowed to cool down to room temperature and then quenched with water (80 ml). The yellow precipitate was isolated by suction and washed with ethanol/water (1:1). The solid was dissolved in ethyl acetate, dried over sodium sulfate and concentrated to yield 4.15 g (93%) 6-amino-1-ethyl-3,3-dimethyl-5-nitro-1,3-dihydro-indol-2-one as a orange solid.

MS: M=250.0 (API+)

¹H-NMR (400 MHz, DMSO): δ (ppm)=1.15 (t, 3H), 1.27 (s, 6H), 3.64 (m, 2H), 6.54 (s, 1H), 7.67 (br, 2H), 7.95 (s, 1H)

### vi) 5,6-Diamino-1-ethyl-3,3-dimethyl-1,3-dihydro-indol-2-one

To a solution of 6-amino-1-ethyl-3,3-dimethyl-5-nitro-1,3-dihydro-indol-2-one (4.15 g, 16.65 mmol) in ethanol (80 ml) PtO₂ (0.4 g) was added and the mixture hydrogenated at room temperature for 3.5 h. After filtration and evaporation of the solvents 3.25 g (89%) 5,6-diamino-1-ethyl-3,3-dimethyl-1,3-dihydro-indol-2-one was isolated as orange solid.

MS: M=220.0 (API+)

¹H-NMR (400 MHz, DMSO): δ (ppm)=1.10 (t, 3H), 1.13 (s, 6H), 3.53 (m, 2H), 4.08 (br, 2H), 4.48 (br, 2H), 6.27 (s, 1H), 6.50 (s, 1H)

### A2. Preparation of 5,6-diamino-1,3,3-trimethyl-1,3-dihydro-indol-2-one

5,6-diamino-1,3,3-trimethyl-1,3-dihydro-indol-2-one was prepared in an analogous 6-step-synthesis as described for 5,6-diamino-1-ethyl-3,3-dimethyl-1,3-dihydro-indol-2-one (A1).

MS: M=206.1 (API+)

¹H-NMR (400 MHz, DMSO): δ (ppm)=1.57 (s, 6H), 3.43 (s, 3H), 4.94 (br, 4H), 6.66 (s, 1H), 6.95 (s, 1H)

### A3. Preparation of 5,6-diamino-3,3-dimethyl-1-1-propyl-1,3-dihydro-indol-2-one

5,6-diamino-3,3-dimethyl-1-propyl-1,3-dihydro-indol-2-one was prepared in an analogous 6-step-synthesis as described for 5,6-diamino-1-ethyl-3,3-dimethyl-1,3-dihydro-indol-2-one (A1).

MS: M=234.1 (API+)

¹H-NMR (400 MHz, DMSO): δ (ppm)=0.82 (t, 3H), 1.15 (s, 6H), 1.58 (m, 2H), 3.46 (q, 2H), 4.16 (br, 2H), 4.45 (br, 2H), 6.27 (s, 1H), 6.50 (s, 1H)

### A4. Preparation of 5,6-diamino-1-isopropyl-3,3-dimethyl-1,3-dihydro-indol-2-one

5,6-diamino-3,3-dimethyl-1-isopropyl-1,3-dihydro-indol-2-one was prepared in an analogous 6-step-synthesis as described for 5,6-diamino-1-ethyl-3,3-dimethyl-1,3-dihydro-indol-2-one (A1).

MS: M=234.1 (API+)

¹H-NMR (400 MHz, DMSO): δ (ppm)=1.12 (s, 6H), 1.33 (d, 6H), 4.09 (br, 2H), 4.40 (m, 1H), 4.46 (br, 2H), 6.46 (s, 1H), 6.48 (s, 1H)

### A5. Preparation of 5,6-diamino-3,3-dimethyl-1-(3-morpholin-4-yl-propyl)-1,3-dihydro-indol-2-one

5,6-diamino-3,3-dimethyl-1-(3-morpholin-4-yl-propyl)-1,3-dihydro-indol-2-one was prepared in an analogous 6-step-synthesis as described for 5,6-diamino-1-ethyl-3,3-dimethyl-1,3-dihydro-indol-2-one (A1).

MS: M=319.1 (API+)

¹H-NMR (400 MHz, DMSO): δ (ppm)=1.14 (s, 6H), 1.70 (m, 2H), 2.26 (t, 2H), 2.33 (m, 4H), 3.56 (m, 6H), 4.39 (br, 4H), 6.28 (s, 1H), 6.50 (s, 1H)

### A6. Preparation of 5,6-diamino-3,3-diethyl-1-isopropyl-1,3-dihydro-indol-2-one

### i) 3,3-Diethyl-5-nitro-1,3-dihydro-indol-2-one

To a solution of 3,3-diethyl-1,3-dihydro-indol-2-one (10.0 g, 52.84 mmol, A. Mertens et al., J. Med. Chem. 1987, 30, 1279-1287) in conc. sulfuric acid (50 ml) was added slowly a mixture of nitric acid (65%, 5.12 g, 3.63 ml, 52.84 mmol) and conc. sulfuric acid (10 ml) at 0° C. After 2 h at room temperature the mixture was poured into ice water. The precipitate was filtered off, washed with water and dried to yield 11.7 g 3,3-diethyl-5-nitro-1,3-dihydro-indol-2-one (49.95 mmol, 94%).

MS: M=235.1 (ESI+)

### ii) 3,3-Diethyl-1-isopropyl-5-nitro-1,3-dihydro-indol-2-one

A solution of 3,3-diethyl-5-nitro-1,3-dihydro-indol-2-one (11.7 g, 49.95 mmol) in anhydrous N,N-dimethylformamide (DMF) (60 ml) was treated with sodium hydride (1.558 g, 64.93 mmol). The resulting suspension was stirred for 1 h at 60° C. A solution of 2-iodo-propane (4.99 ml, 8.49 g, 49.95 mmol) was added. The mixture was kept at 60° C. for further 3 h, allowed to cool to room temperature poured into ice water. The precipitate was filtered off, washed with water and dried to yield 12.6 g 3,3-diethyl-1-isopropyl-5-nitro-1,3-dihydro-indol-2-one (45.60 mmol, 91%)

MS: M=277.1 (ESI+)

### iii) 5-Amino-3,3-diethyl-1-isopropyl-1,3-dihydro-indol-2-one

To a solution of 3,3-diethyl-1-isopropyl-5-nitro-1,3-dihydro-indol-2-one (12.6 g, 45.60 mmol) in methanol/tetrahydrofuran (THF) (1:1, 80 ml) palladium on charcoal (10%, 1.2 g) was added and the mixture hydrogenated at room temperature for 4 h. After filtration of the catalyst the solvent was evaporated and the residue triturated with iso-hexane to yield 9.7 g 5-amino-3,3-diethyl-1-isopropyl-1,3-dihydro-indol-2-one (39.37 mmol, 86%).

MS: M=247.1 (ESI+)

### iv) N-(3,3-Diethyl-1-isopropyl-2-oxo-2,3-dihydro-1H-indol-5-yl)-acetamide

A solution of 5-amino-3,3-diethyl-1-isopropyl-1,3-dihydro-indol-2-one (9.7 g, 39.37 mmol) in acetic anhydride (57 ml) was stirred at room temperature for 4 h. The mixture was poured into ice water, allowed to warm to room temperature and was stirred again for 2 h. After extraction with ethyl acetate, the combined organic layers were washed with aqueous NaOH solution (1M) and brine and dried over sodium sulfate. After removal of the solvent the crude product was triturated with iso-hexane to yield 10.4 g N-(3,3-Diethyl-1-isopropyl-2-oxo-2,3-dihydro-1H-indol-5-yl)-acetamide (36.06 mmol, 91%)

MS: M=289.2 (ESI+)

### v) N-(3,3-Diethyl-1-isopropyl-6-nitro-2-oxo-2,3-dihydro-1H-indol-5-yl)-acetamide

To a solution of N-(3,3-diethyl-1-isopropyl-2-oxo-2,3-dihydro-1H-indol-5-yl)-acetamide (10.4 g, 36.06 mmol) in conc. sulfuric acid (50 ml) was added slowly a mixture of nitric acid (65%, 3.84 g, 2.72 ml, 39.67 mmol) and conc. sulfuric acid (10 ml) at 0° C. After 2 h at room temperature the mixture was poured into ice water. The precipitate was filtered off, washed with water and dried. The crude material was purified by silica gel chromatography (isohexane/ethyl acetate 1:1) to yield 2.2 g N-(3,3-diethyl-1-isopropyl-6-nitro-2-oxo-2,3-dihydro-1H-indol-5-yl)-acetamide (6.60 mmol, 18%) besides undesired N-(3,3-diethyl-1-isopropyl-7-nitro-2-oxo-2,3-dihydro-1H-indol-5-yl)-acetamide (5.5 g).

MS: M=332.2 (ESI−)

### vi) 5-Amino-3,3-diethyl-1-isopropyl-6-nitro-1,3-dihydro-indol-2-one

N-(3,3-diethyl-1-isopropyl-6-nitro-2-oxo-2,3-dihydro-1H-indol-5-yl)-acetamide (2.2 g, 6.60 mmol) was dissolved in ethanol (50 ml). After addition of hydrochloric acid (25%, 3.2 ml, 33.0 mmol) the mixture was heated under reflux for 3 h. Most of the solvent was evaporated and water was added. The mixture was weakly alkalized by addition of aqueous NaOH solution. The mixture was extracted with ethyl acetate, the combined organic phases were dried over magnesium sulfate and the solvent was evaporated to yield 1.9 g 5-amino-3,3-diethyl-1-isopropyl-6-nitro-1,3-dihydro-indol-2-one (6.52 mmol, 99%).

MS: M=290.1 (ESI−)

### vii) 5,6-Diamino-3,3-diethyl-1-isopropyl-1,3-dihydro-indol-2-one

To a solution of 5-amino-3,3-diethyl-1-isopropyl-6-nitro-1,3-dihydro-indol-2-one (1.9 g, 6.52 mmol) in methanol/tetrahydrofuran (THF) (1:1, 80 ml) palladium on charcoal (10%, 1.2 g) was added and the mixture hydrogenated at room temperature for 4 h. After filtration the solvent was evaporated and the residue triturated with iso-hexane to yield 1.7 g 5,6-diamino-3,3-diethyl-1-isopropyl-1,3-dihydro-indol-2-one (6.50 mmol, 99%).

MS: M=262.3 (ESI+)

¹H-NMR (400 MHz, DMSO): δ (ppm)=0.44 (t, 6H), 1.34 (d, 6H), 1.55 (q, 2H), 1.65 (q, 2H), 4.40 (br, 4H), 4.45 (m, 1H), 6.42 (s, 1H), 6.46 (s, 1H)

### A7. Preparation of 5,6-diamino-1,3,3-triethyl-1,3-dihydro-indol-2-one

5,6-Diamino-1,3,3-triethyl-1,3-dihydro-indol-2-one was prepared in an analogous 7-step-synthesis as described for 5,6-diamino-3,3-diethyl-1-isopropyl-1,3-dihydro-indol-2-one (A6).

MS: M=248.1 (API+)

¹H-NMR (400 MHz, DMSO): δ (ppm)=0.43 (t, 6H), 1.08 (t, 3H), 1.55 (q, 2H), 1.63 (q, 2H), 3.54 (q, 2H), 4.10 (br, 2H), 4.48 (br, 2H), 6.27 (s, 1H), 6.43 (s, 1H)

### A8. Preparation of 5-Benzyl-4,5,6,7-tetrahydro-1H-pyrazolo[4,3-c]pyridine-3-carboxylic acid ethyl ester

### i) (1-Benzyl-4-oxo-piperidin-3-yl)-oxo-acetic acid ethyl ester

Sodium (1.340 g, 58.28 mmol) was added to ice-cooled ethanol (50 ml) under a nitrogen atmosphere. After 15 h at 0° C., the solution was cooled to −10° C. and oxalic acid diethyl ester (7.722 g, 52.84 mmol) was added dropwise. Then a solution of 1-benzyl-piperidin-4-one (9.995 g, 52.81 mmol) in ethanol (30 ml) was added within an hour. The reaction mixture was warmed to room temperature and after 5 h the solvent was evaporated to give crude (1-benzyl-4-oxo-piperidin-3-yl)-oxo-acetic acid ethyl ester (17.07 g) which was used for the next reaction without further purification.

MS: M=290.1 (ESI+)

### ii) 5-Benzyl-4,5,6,7-tetrahydro-1H-pyrazolo[4,3-c]pyridine-3-carboxylic acid ethyl ester

To a solution of (1-benzyl-4-oxo-piperidin-3-yl)-oxo-acetic acid ethyl ester (17.07 g) in acetic acid (60 ml) at 0° C. was added hydrazine hydrate (2.959 g, 59.10 mmol). After heating to 120° C. under reflux for 6 h the reaction mixture was cooled to room temperature and treated with water (150 ml) and ethyl acetate (150 ml). The organic phase was washed with saturated aqueous bicarbonate solution until pH 7-8. The combined aqueous phases were reextracted twice with ethyl acetate and the combined organic phases were dried over MgSO4. The solvent was evaporated and the residue subjected to silica gel chromatography (0.5% triethylamine in ethyl acetate) to yield 3.00 g 5-benzyl-4,5,6,7-tetrahydro-1H-pyrazolo[4,3-c]pyridine-3-carboxylic acid ethyl ester (10.51 mmol, 17.8%)

MS: M=286.1 (API+)

### A9. Preparation of 5-Benzyl-1,4,5,6-tetrahydro-pyrrolo[3,4-c]pyrazole-3-carboxylic acid ethyl ester

### i) (1-Benzyl-4-oxo-pyrrolidin-3-yl)-oxo-acetic acid ethyl ester

In an analogous manner as described for A8 i) (1-benzyl-4-oxo-pyrrolidin-3-yl)-oxo-acetic acid ethyl ester was prepared from 1-benzyl-3-pyrrolidinone.

MS: M=276.1 (API+)

### ii) 5-Benzyl-1,4,5,6-tetrahydro-pyrrolo[3,4-c]pyrazole-3-carboxylic acid ethyl ester

In an analogous manner as described for A8 ii) 5-benzyl-1,4,5,6-tetrahydro-pyrrolo[3,4-c]pyrazole-3-carboxylic acid ethyl ester was prepared from (1-benzyl-4-oxo-pyrrolidin-3-yl)-oxo-acetic acid ethyl ester.

MS: M=272.0 (API+)

### A10. Preparation of 1,4,5,6-Tetrahydro-pyrrolo[3,4-c]pyrazole-3-carboxylic acid ethyl ester

To a solution of 5-benzyl-1,4,5,6-tetrahydro-pyrrolo[3,4-c]pyrazole-3-carboxylic acid ethyl ester (A9, 2.74 g, 10.099 mmol) in ethanol/tetrahydrofuran (THF) (1:2, 60 ml) palladium on charcoal (10%, 0.55 g) was added and the mixture hydrogenated at room temperature and 44 mbar for 4.5 h. After filtration of the catalyst the solvent was evaporated and the residue was recrystallized from diethyl ether to yield 1.50 g 1,4,5,6-tetrahydro-pyrrolo[3,4-c]pyrazole-3-carboxylic acid ethyl ester (8.278 mmol, 82%).

MS: M=182.2 (ESI+)

### A11. Preparation of 5-(2,6-Diethyl-phenylcarbamoyl)-1,4,5,6-tetrahydro-pyrrolo[3,4-c]pyrazole-3-carboxylic acid

### i) 5-(2,6-Diethyl-phenylcarbamoyl)-1,4,5,6-tetrahydro-pyrrolo[3,4-c]pyrazole-3-carboxylic acid ethyl ester

To a solution of 1,4,5,6-tetrahydro-pyrrolo[3,4-c]pyrazole-3-carboxylic acid ethyl ester (A10, 200 mg, 1.104 mmol) in DMF (3 ml) was added triethylamine (223.4 mg, 308 μl, 2.208 mmol) and a solution of 1,3-diethyl-2-isocyanato-benzene (193.4 mg, 191 μl, 1.104 mmol) in DMF (1 ml). After 1 h at room temperature the mixture was treated with water (40 ml). The aqueous phase was extracted with ethyl acetate, the combined organic phases were dried over MgSO₄ and the solvent was evaporated. The residue was purified by silica gel chromatography (ethyl acetate/heptan 2:1) to yield 304 mg 5-(2,6-diethyl-phenylcarbamoyl)-1,4,5,6-tetrahydro-pyrrolo[3,4-c]pyrazole-3-carboxylic acid ethyl ester (0.853 mmol, 77%).

MS: M=357.2 (ESI+)

### ii) 5-(2,6-Diethyl-phenylcarbamoyl)-1,4,5,6-tetrahydro-pyrrolo[3,4-c]pyrazole-3-carboxylic acid

To a solution of 5-(2,6-diethyl-phenylcarbamoyl)-1,4,5,6-tetrahydro-pyrrolo[3,4-c]pyrazole-3-carboxylic acid ethyl ester (245 mg, 0.687 mmol) in THF (4 ml) was added NaOH (2N, 2 ml, 4.0 mmol) and heated under reflux for 2 h. The mixture was cooled to room temperature and acidified with HCl (2N). The aqueous phase was extracted ethyl acetate, the combined organic phases were washed with brine, dried over MgSO₄ and the solvent was evaporated to yield 225 mg 5-(2,6-diethyl-phenylcarbamoyl)-1,4,5,6-tetrahydro-pyrrolo[3,4-c]pyrazole-3-carboxylic acid (0.685 mmol, 99%).

MS: M=329.1 (ESI+)

### A12. Preparation of 5-Cyclopropanecarbonyl-1,4,5,6-tetrahydro-pyrrolo[3,4-c]pyrazole-3-carboxylic acid

### i) 5-Cyclopropanecarbonyl-1,4,5,6-tetrahydro-pyrrolo[3,4-c]pyrazole-3-carboxylic acid ethyl ester

To a solution of 1,4,5,6-tetrahydro-pyrrolo[3,4-c]pyrazole-3-carboxylic acid ethyl ester (A10, 100 mg, 0.552 mmol) in THF (2 ml) were added cyclopropanecarbonyl chloride (63.5 mg, 0.607 mmol) and diisopropylethylamine (178.3 mg, 240 μl, 1.380 mmol) at 0° C. After stirring at room temperature for 12 h the mixture was quenched with NaOH (2N, 0.5 ml) and water (5 ml). The aqueous phase was extracted ethyl acetate, the combined organic phases were dried over Na₂SO₄ and the solvent was evaporated to yield 98 mg 5-cyclopropanecarbonyl-1,4,5,6-tetrahydro-pyrrolo[3,4-c]pyrazole-3-carboxylic acid ethyl ester (0.393 mmol, 71%)

MS: M=250.0 (API+)

### ii) 5-Cyclopropanecarbonyl-1,4,5,6-tetrahydro-pyrrolo[3,4-c]pyrazole-3-carboxylic acid

In an analogous manner as described for A11 ii) 5-cyclopropanecarbonyl-1,4,5,6-tetrahydro-pyrrolo[3,4-c]pyrazole-3-carboxylic acid was prepared from 5-cyclopropanecarbonyl-1,4,5,6-tetrahydro-pyrrolo[3,4-c]pyrazole-3-carboxylic acid ethyl ester.

### A13. 5-(2-Thiophen-2-yl-acetyl)-1,4,5,6-tetrahydro-pyrrolo[3,4-c]pyrazole-3-carboxylic acid

### i) 5-(2-Thiophen-2-yl-acetyl)-1,4,5,6-tetrahydro-pyrrolo[3,4-c]pyrazole-3-carboxylic acid ethyl ester

In an analogous manner as described for A12 i) 5-(2-thiophen-2-yl-acetyl)-1,4,5,6-tetrahydro-pyrrolo[3,4-c]pyrazole-3-carboxylic acid ethyl ester was prepared from 1,4,5,6-tetrahydro-pyrrolo[3,4-c]pyrazole-3-carboxylic acid ethyl ester (A10) and thiophen-2-yl-acetyl chloride.

MS: M=306.2 (API+)

### ii) 5-(2-Thiophen-2-yl-acetyl)-1,4,5,6-tetrahydro-pyrrolo[3,4-c]pyrazole-3-carboxylic acid

In an analogous manner as described for A11 ii) 5-(2-thiophen-2-yl-acetyl)-1,4,5,6-tetrahydro-pyrrolo[3,4-c]pyrazole-3-carboxylic acid was prepared from 5-(2-thiophen-2-yl-acetyl)-1,4,5,6-tetrahydro-pyrrolo[3,4-c]pyrazole-3-carboxylic acid ethyl ester.

MS: M=278.1 (ESI+)

### A14. 5-(4-Fluoro-benzenesulfonyl)-1,4,5,6-tetrahydro-pyrrolo[3,4-c]pyrazole-3-carboxylic acid

### i) 5-(4-Fluoro-benzenesulfonyl)-1,4,5,6-tetrahydro-pyrrolo[3,4-c]pyrazole-3-carboxylic acid ethyl ester

In an analogous manner as described for A12 i) 5-(4-Fluoro-benzenesulfonyl)-1,4,5,6-tetrahydro-pyrrolo[3,4-c]pyrazole-3-carboxylic acid ethyl ester was prepared from 1,4,5,6-tetrahydro-pyrrolo[3,4-c]pyrazole-3-carboxylic acid ethyl ester (A10) and 4-fluoro-benzenesulfonyl chloride.

MS: M=340.0 (ESI+)

### ii) 5-(4-Fluoro-benzenesulfonyl)-1,4,5,6-tetrahydro-pyrrolo[3,4-c]pyrazole-3-carboxylic acid

In an analogous manner as described for A11 ii) 5-(4-fluoro-benzenesulfonyl)-1,4,5,6-tetrahydro-pyrrolo[3,4-c]pyrazole-3-carboxylic acid was prepared from 5-(4-fluoro-benzenesulfonyl)-1,4,5,6-tetrahydro-pyrrolo[3,4-c]pyrazole-3-carboxylic acid ethyl ester.

MS: M=310.0 (ESI−)

### A15. 1,4,6,7-Tetrahydro-thiopyrano[4,3-c]pyrazole-3-carboxylic acid

### i) Oxo-(4-oxo-tetrahydro-thiopyran-3-yl)-acetic acid ethyl ester

In an analogous manner as described for A8 i) oxo-(4-oxo-tetrahydro-thiopyran-3-yl)-acetic acid ethyl ester was prepared from tetrahydro-thiopyran-4-one.

MS: M=215.0 (API−)

### ii) 1,4,6,7-Tetrahydro-thiopyrano[4,3-c]pyrazole-3-carboxylic acid ethyl ester

In an analogous manner as described for A8 ii) 1,4,6,7-tetrahydro-thiopyrano[4,3-c]pyrazole-3-carboxylic acid ethyl ester was prepared from oxo-(4-oxo-tetrahydro-thiopyran-3-yl)-acetic acid ethyl ester.

MS: M=213.1 (ESI+)

### iii) 1,4,6,7-Tetrahydro-thiopyrano[4,3-c]pyrazole-3-carboxylic acid

In an analogous manner as described for A11 ii) 1,4,6,7-tetrahydro-thiopyrano[4,3-c]pyrazole-3-carboxylic acid was prepared from 1,4,6,7-tetrahydro-thiopyrano[4,3-c]pyrazole-3-carboxylic acid ethyl ester.

MS: M=183.1 (ESI−)

### A15. 4,6-Dihydro-1H-thieno[3,4-c]pyrazole-3-carboxylic acid

### i) Oxo-(4-oxo-tetrahydro-thiophen-3-yl)-acetic acid ethyl ester

In an analogous manner as described for A8 i) oxo-(4-oxo-tetrahydro-thiophen-3-yl)-acetic acid ethyl ester was prepared from dihydro-thiophen-3-one.

### ii) 4,6-Dihydro-1H-thieno[3,4-c]pyrazole-3-carboxylic acid ethyl ester

In an analogous manner as described for A8 ii) 4,6-dihydro-1H-thieno[3,4-c]pyrazole-3-carboxylic acid ethyl ester was prepared from oxo-(4-oxo-tetrahydro-thiophen-3-yl)-acetic acid ethyl ester.

MS: M=199.0 (API+)

### iii) 4,6-Dihydro-1H-thieno[3,4-c]pyrazole-3-carboxylic acid

In an analogous manner as described for A11 ii) 4,6-Dihydro-1H-thieno[3,4-c]pyrazole-3-carboxylic acid was prepared from 4,6-dihydro-1H-thieno[3,4-c]pyrazole-3-carboxylic acid ethyl ester.

MS: M=171.0 (API+)

### Final Products

### EXAMPLE 1

### 7,7-Dimethyl-2-(1,4,5,6-tetrahydro-cyclopentapyrazol-3-yl)-5,7-dihydro-3H-imidazo[4,5-f]indol-6-one

5,6-Diamino-3,3-dimethyl-1,3-dihydro-indol-2-one (143 mg, 0.75 mmol), and 1,4,5,6-Tetrahydro-cyclopentapyrazole-3-carboxylic acid (114 mg, 0.75 mmol) were mixed with polyphosphoric acid (5.10 g, 53.12 mmol) and phosphorus pentoxide (190 mg, 1.34 mmol) and stirred under nitrogen at 150° C. for 6 h. The mixture was quenched with ice water (25 ml) and the resulting solution was adjusted to pH 7-8 by adding aqueous ammonia and then extracted twice with ethyl acetate (3×50 ml). The combined organic layers were washed with water (50 ml), dried over sodium sulfate and concentrated. The crude product was purified by HPL chromatography. Yield 37 mg (16%) of a light brown solid.

MS: M=308.1 (API+)

¹H-NMR (400 MHz, D₆-DMSO): δ (ppm)=1.29 (s, 6H), 2.52 (m, 2H), 2.71 (m, 2H), 2.81 (m, 2H), 6.88 (br, 1H), 6.95 (br, 1H), 10.23 (br, 1H)

### EXAMPLE 2

### 5,7,7-Trimethyl-2-(1,4,5,6-tetrahydro-cyclopentapyrazol-3-yl)-5,7-dihydro-3H-imidazo[4,5-f]indol-6-one

In an analogous manner as described for example 1 5,7,7-Trimethyl-2-(1,4,5,6-tetrahydro-cyclopentapyrazol-3-yl)-5,7-dihydro-3H-imidazo[4,5-f]indol-6-one was prepared from the appropriate starting material.

MS: M=322.0 (API+)

¹H-NMR (400 MHz, D₆-DMSO): δ (ppm)=1.31 (s, 6H), 2.52 (m, 2H), 2.71 (m, 2H), 2.81 (m, 2H), 3.19 (s, 3H), 6.95 and 7.23 (s, 1H, two tautomeric forms), 7.39 and 7.61 (s, 1H, two tautomeric forms), 12.48 (br, 1H), 12.70 (br, 1H)

### EXAMPLE 3

### 5-Ethyl-7,7-dimethyl-2-(1,4,5,6-tetrahydro-cyclopentapyrazol-3-yl)-5,7-dihydro-3H-imidazo[4,5-f]indol-6-one

In an analogous manner as described for example 15-Ethyl-7,7-dimethyl-2-(1,4,5,6-tetrahydro-cyclopentapyrazol-3-yl)-5,7-dihydro-3H-imidazo[4,5-f]indol-6-one was prepared from the appropriate starting material.

MS: M=336.2 (API+)

¹H-NMR (400 MHz, D₆-DMSO): δ (ppm)=1.18 (t, 3H), 1.31 (s, 6H), 2.55 (m, 2H), 2.70 (m, 2H), 2.81 (m, 2H), 3.76 (q, 2H), 6.98 and 7.27 (s, 1H, two tautomeric forms), 7.37 and 7.61 (s, 1H, two tautomeric forms), 12.50 (br, 1H), 12.75 (br, 1H)

### EXAMPLE 4

### 7,7-Dimethyl-5-propyl-2-(1,4,5,6-tetrahydro-cyclopentapyrazol-3-yl)-5,7-dihydro-3H-imidazo[4,5-f]indol-6-one

In an analogous manner as described for example 1 7,7-Dimethyl-5-propyl-2-(1,4,5,6-tetrahydro-cyclopentapyrazol-3-yl)-5,7-dihydro-3H-imidazo[4,5-f]indol-6-one was prepared from the appropriate starting material.

MS: M=350.1 (API+)

¹H-NMR (400 MHz, D₆-DMSO): δ (ppm)=0.85 (m, 3H), 1.31 (s, 6H), 1.66 (m, 2H), 2.52 (m, 2H), 2.71 (m, 2H), 2.81 (m, 2H), 3.69 (t, 2H), 6.98 and 7.27 (s, 1H, two tautomeric forms), 7.37 and 7.61 (s, 1H, two tautomeric forms), 12.41 an 12.50 (br, 1H, two tautomeric forms), 12.70 (br, 1H)

### EXAMPLE 5

### 5-Isopropyl-7,7-dimethyl-2-(1,4,5,6-tetrahydro-cyclopentapyrazol-3-yl)-5,7-dihydro-3H-imidazo[4,5-f]indol-6-one

In an analogous manner as described for example 15-Isopropyl-7,7-dimethyl-2-(1,4,5,6-tetrahydro-cyclopentapyrazol-3-yl)-5,7-dihydro-3H-imidazo[4,5-f]indol-6-one was prepared from the appropriate starting material.

MS: M=350.1 (API+)

¹H-NMR (400 MHz, D₆-DMSO): δ (ppm)=1.29 (s, 6H), 1.44 (d, 6H), 2.51 (m, 2H), 2.71 (m, 2H), 2.81 (m, 2H), 4.57 (m, 1H), 7.1-7.6 (br, tautomeric forms, 2H), 12.75 (br, 2H)

### EXAMPLE 6

### 7,7-Dimethyl-5-(3-morpholin-4-yl-propyl)-2-(1,4,5,6-tetrahydro-cyclopentapyrazol-3-yl)-5,7-dihydro-3H-imidazo[4,5-f]indol-6-one

In an analogous manner as described for example 1 7,7-Dimethyl-5-(3-morpholin-4-yl-propyl)-2-(1,4,5,6-tetrahydro-cyclopentapyrazol-3-yl)-5,7-dihydro-3H-imidazo[4,5-f]indol-6-one was prepared from the appropriate starting material.

MS: M=435.2 (API+)

¹H-NMR (400 MHz, D₆-DMSO): δ (ppm)=1.31 (s, 6H), 1.78 (m, 2H), 2.29 (m, 6H), 2.52 (m, 2H), 2.70 (m, 2H), 2.81 (m, 2H), 3.58 (m, 4H), 3.75 (t, 2H), 6.02 and 7.29 (s, 1H, two tautomeric forms), 7.37 and 7.58 (s, 1H, two tautomeric forms), 12.41 and 12.50 (br, 2H)

### EXAMPLE 7

### 7,7-Dimethyl-2-(4,5,6,7-tetrahydro-1H-indazol-3-yl)-5,7-dihydro-3H-imidazo[4,5-f]indol-6-one

In an analogous manner as described for example 1 7,7-Dimethyl-2-(4,5,6,7-tetrahydro-1H-indazol-3-yl)-5,7-dihydro-3H-imidazo[4,5-f]indol-6-one was prepared from the appropriate starting material.

MS: M=322.0 (API+)

¹H-NMR (400 MHz, D₆-DMSO): δ (ppm)=1.29 (s, 6H), 1.76 (m, 4H), 2.63 (m, 2H), 2.81 (m, 2H), 6.88 and 6.99 (s, 1H, two tautomeric forms), 7.28 and 7.52 (s, 1H, two tautomeric forms), 10.18 and 10.24 (br, 1H, two tautomeric forms), 12.32 and 12.46 (br, 1H), 12.72 and 12.74 (br, 1H, two tautomeric forms)

### EXAMPLE 8

### 5-Ethyl-7,7-dimethyl-2-(4,5,6,7-tetrahydro-1H-indazol-3-yl)-5,7-dihydro-3H-imidazo[4,5-f]indol-6-one

In an analogous manner as described for example 15-Ethyl-7,7-dimethyl-2-(4,5,6,7-tetrahydro-1H-indazol-3-yl)-5,7-dihydro-3H-imidazo[4,5-f]indol-6-one was prepared from the appropriate starting material.

MS: M=350.1 (API+)

¹H-NMR (400 MHz, D₆-DMSO): δ (ppm)=1.19 (t, 3H), 1.30 (s, 6H), 1.76 (m, 4H), 2.63 (m, 2H), 2.82 (m, 2H), 3.75 (q, 2H), 6.95 and 7.29 (s, 1H, two tautomeric forms), 7.35 and 7.63 (s, 1H, two tautomeric forms), 12.48 and 12.53 (br, 1H), 12.75 (br, 1H)

### EXAMPLE 9

### 5-Isopropyl-7,7-dimethyl-2-(4,5,6,7-tetrahydro-1H-indazol-3-yl)-5,7-dihydro-3H-imidazo[4,5-f]indol-6-one

In an analogous manner as described for example 15-Isopropyl-7,7-dimethyl-2-(4,5,6,7-tetrahydro-1H-indazol-3-yl)-5,7-dihydro-3H-imidazo[4,5-f]indol-6-one was prepared from the appropriate starting material.

MS: M=364.1 (API+)

¹H-NMR (400 MHz, D₆-DMSO): δ (ppm)=1.28 (s, 6H), 1.45 (d, 6H), 1.76 (m, 4H), 2.64 (m, 2H), 2.82 (m, 2H), 4.55 (m, 1H), 7.08 and 7.33 (s, 1H, two tautomeric forms), 7.35 and 7.60 (s, 1H, two tautomeric forms), 12.40 and 12.52 (br, 1H), 12.75 (br, 1H)

### EXAMPLE 10

### 7,7-Dimethyl-5-(3-morpholin-4-yl-propyl)-2-(4,5,6,7-tetrahydro-1H-indazol-3-yl)-5,7-dihydro-3H-imidazo[4,5-f]indol-6-one

In an analogous manner as described for example 1 7,7-Dimethyl-5-(3-morpholin-4-yl-propyl)-2-(4,5,6,7-tetrahydro-1H-indazol-3-yl)-5,7-dihydro-3H-imidazo[4,5-f]indol-6-one was prepared from the appropriate starting material.

MS: M=449.1 (API+)

¹H-NMR (400 MHz, D₆-DMSO): δ (ppm)=1.30 (s, 6H), 1.78 (m, 6H), 2.29 (m, 6H), 2.64 (m, 2H), 2.81 (m, 2H), 3.58 (m, 4H), 3.74 (t, 2H), 7.00 and 7.31 (s, 1H, two tautomeric forms), 7.34 and 7.59 (s, 1H, two tautomeric forms), 12.51 and 12.53 (br, 1H, two tautomeric forms), 12.74 and 12.76 (br, 1H, two tautomeric forms)

### EXAMPLE 11

### 5-Ethyl-7,7-dimethyl-2-(1,4,6,7-tetrahydro-pyrano[4,3-c]pyrazol-3-yl)-5,7-dihydro-3H-imidazo[4,5-f]indol-6-one

In an analogous manner as described for example 1 7,7-Dimethyl-5-(3-morpholin-4-yl-propyl)-2-(4,5,6,7-tetrahydro-1H-indazol-3-yl)-5,7-dihydro-3H-imidazo[4,5-f]indol-6-one was prepared from the appropriate starting material.

MS: M=352.1 (API+)

¹H-NMR (400 MHz, DMSO): δ (ppm)=1.60 (t, 3H), 1.72 (s, 6H), 3.19 (t, 2H), 4.17 (q, 2H), 4.30 (t, 2H), 5.31 (s, 2H), 7.39 and 7.71 (s, 1H, two tautomeric forms), 7.79 and 8.05 (s, 1H, two tautomeric forms), 13.08 and 13.13 (br, 1H, two tautomeric forms), 13.43 and 13.45 (br, 1H, two tautomeric forms)

### EXAMPLE 12

### 5,7,7-Triethyl-2-(4,5,6,7-tetrahydro-1H-indazol-3-yl)-5,7-dihydro-3H-imidazo[4,5-f]indol-6-one

In an analogous manner as described for example 15,7,7-triethyl-2-(4,5,6,7-tetrahydro-1H-indazol-3-yl)-5,7-dihydro-3H-imidazo[4,5-f]indol-6-one was prepared from the appropriate starting material.

MS: M=378.3 (ESI+)

¹H-NMR (400 MHz, D₆-DMSO): δ (ppm)=0.45 (t, 6H), 1.16 (t, 3H), 1.81 (m, 8H), 2.65 (t, 2H), 2.83 (t, 2H), 3.77 (q, 2H), 6.95 and 7.25 (s, 1H, two tautomeric forms), 7.27 and 7.50 (s, 1H, two tautomeric forms), 12.50 and 12.53 (br, 1H, two tautomeric forms, 12.75 and 12.78 (br, 1H, two tautomeric forms)

### EXAMPLE 13

### 2-(5-Benzyl-4,5,6,7-tetrahydro-1H-pyrazolo[4,3-c]pyridin-3-yl)-5-ethyl-7,7-dimethyl-5,7-dihydro-3H-imidazo[4,5-f]indol-6-one

5,6-Diamino-1-ethyl-3,3-dimethyl-1,3-dihydro-indol-2-one (A1, 1.109 g, 5.057 mmol), and 5-benzyl-4,5,6,7-tetrahydro-1H-pyrazolo[4,3-c]pyridine-3-carboxylic acid ethyl ester (A8, 1.44 g, 5.046 mmol) were mixed with polyphosphoric acid (16.82 g, 117.6 mmol) and phosphorous pentoxide (4.305 g, 30.33 mmol) and stirred under nitrogen at 150° C. for 12 h. The mixture was quenched with ice water (70 ml) and the resulting solution was adjusted to pH 7-8 by adding aqueous ammonia. After 3 h in the refrigerator the precipitate was filtered off and purified by HPL chromatography to yield 580 mg 2-(5-benzyl-4,5,6,7-tetrahydro-1H-pyrazolo[4,3-c]pyridin-3-yl)-5-ethyl-7,7-dimethyl-5,7-dihydro-3H-imidazo[4,5-f]indol-6-one (1.316 mmol, 26%)

MS: M=441.1 (API+)

¹H-NMR (400 MHz, DMSO): δ (ppm)=1.13 (m, 3H), 1.28 (s, 6H), 2.76 (m, 4H), 3.70-3.84 (m, 6H), 6.95 and 7.29 (s, 1H, two tautomeric forms), 7.27 (m, 1H), 7.31-7.40 (m, 4H), 7.41 and 7.60 (s, 1H, two tautomeric forms), 12.55 and 12.61 (s, 1H, two tautomeric forms)

### EXAMPLE 14

### 5-Ethyl-7,7-dimethyl-2-(4,5,6,7-tetrahydro-1H-pyrazolo[4,3-c]pyridin-3-yl)-5,7-dihydro-3H-imidazo[4,5-f]indol-6-one

To a solution of 2-(5-benzyl-4,5,6,7-tetrahydro-1H-pyrazolo[4,3-c]pyridin-3-yl)-5-ethyl-7,7-dimethyl-5,7-dihydro-3H-imidazo[4,5-f]indol-6-one (example 13, 76 mg, 0.172 mmol) in methanol/tetrahydrofuran (THF) (1:3, 12 ml) palladium on charcoal (10%, 30 mg) was added and the mixture hydrogenated at room temperature and 32 mbar for 4 h. After filtration of the catalyst the solvent was evaporated to yield 39.3 mg 5-ethyl-7,7-dimethyl-2-(4,5,6,7-tetrahydro-1H-pyrazolo[4,3-c]pyridin-3-yl)-5,7-dihydro-3H-imidazo[4,5-f]indol-6-one (0.112 mmol, 65%)

MS: M=351.1 (API+)

¹H-NMR (400 MHz, DMSO): δ (ppm)=1.18 (t, 3H), 1.30 (s, 6H), 2.69(t, 2H), 3.04 (t, 2H), 3.69-3.81 (m, 2H), 4.08 (s, 2H), 6.96 and 7.28 (s, 1H, two tautomeric forms), 7.37 and 7.61 (s, 1H, two tautomeric forms), 12.51-12.98 (s, 2H)

### EXAMPLE 15

### 5-Ethyl-2-{5-[2-(4-fluoro-phenyl)-acetyl]-4,5,6,7-tetrahydro-1H-pyrazolo[4,3-c]pyridin-3-yl}-7,7-dimethyl-5,7-dihydro-3H-imidazo[4,5-f]indol-6-one

To a solution of (4-fluoro-phenyl)-acetic acid (24 mg, 0.156 mmol) in absolute DMF (1 ml) under a nitrogen atmosphere were added N′-(3-dimethylaminopropyl)-N-ethylcarbodiimide hydrochloride (33 mg, 0.172 mmol) and hydroxybenzotriazole hydrate (26 mg, 0.170 mmol). After 30 minutes at room temperature 5-ethyl-7,7-dimethyl-2-(4,5,6,7-tetrahydro-1H-pyrazolo[4,3-c]pyridin-3-yl)-5,7-dihydro-3H-imidazo[4,5-f]indol-6-one (example 14, 50 mg, 0.143 mmol) was added and stirring continued for 2 h. The reaction mixture was treated with water and the aqueous phase extracted twice with ethyl acetate. The combined organic phases were washed with bicarbonate solution, dried over MgSO₄ and the solvent was evaporated. The residue was purified by HPL chromatography to yield 31.2 mg 5-ethyl-2-{5-[2-(4-fluoro-phenyl)-acetyl]-4,5,6,7-tetrahydro-1H-pyrazolo[4,3-c]pyridin-3-yl}-7,7-dimethyl-5,7-dihydro-3H-imidazo[4,5-f]indol-6-one (0.057 mmol, 40%).

MS: M=487.0 (API+)

¹H-NMR (400 MHz, DMSO): δ (ppm)=1.18 (t, 3H), 1.30 (s, 6H), 2.62-2.77 (d, 2H), 3.67-3.93 (m, 6H), 4.76-4.95 (d, 2H), 6.97 and 7.31 (s, 1H, two tautomeric forms), 7.04-7.20 (m, 2H), 7.22-7.35 (m, 2H), 7.37 and 7.69 (s, 1H, two tautomeric forms), 12.56-12.85 (s, 1H), 12.88-13.19 (s, 1H)

### EXAMPLE 16

### 5-Ethyl-2-[5-(4-fluoro-benzoyl)-4,5,6,7-tetrahydro-1H-pyrazolo[4,3-c]pyridin-3-yl]-7,7-dimethyl-5,7-dihydro-3H-imidazo[4,5-f]indol-6-one

In an analogous manner as described for example 15 5-ethyl-2-[5-(4-fluoro-benzoyl)-4,5,6,7-tetrahydro-1H-pyrazolo[4,3-c]pyridin-3-yl]-7,7-dimethyl-5,7-dihydro-3H-imidazo[4,5-f]indol-6-one was prepared from 5-ethyl-7,7-dimethyl-2-(4,5,6,7-tetrahydro-1H-pyrazolo[4,3-c]pyridin-3-yl)-5,7-dihydro-3H-imidazo[4,5-f]indol-6-one (example 14) and 4-fluoro-benzoic acid.

MS: M=471.2 (API−)

¹H-NMR (400 MHz, DMSO): δ (ppm)=1.18 (m, 3H), 1.30 (s, 6H), 2.85 (m, 2H), 3.51 (m, 2H), 3.75 (m, 2H), 4.93 (m, 2H), 6.96 and 7.37 (s, 1H, two tautomeric forms), 7.31 (t, 2H), 7.41 and 7.73 (s, 1H, two tautomeric forms), 7.57 (m, 2H), 12.53-12.86 (s, 1H), 13.07 and 13.11 (s, 1H, two tautomeric forms)

### EXAMPLE 17

### 5-Ethyl-2-[5-(4-fluoro-benzenesulfonyl)-4,5,6,7-tetrahydro-1H-pyrazolo[4,3-c]pyridin-3-yl]-7,7-dimethyl-5,7-dihydro-3H-imidazo[4,5-f]indol-6-one

To a solution of 5-ethyl-7,7-dimethyl-2-(4,5,6,7-tetrahydro-1H-pyrazolo[4,3-c]pyridin-3-yl)-5,7-dihydro-3H-imidazo[4,5-f]indol-6-one (example 14, 60 mg, 0.171 mmol) in absolute THF (1 ml) at 0° C. were added 4-fluoro-benzenesulfonyl chloride (37 mg, 0.188 mmol) and diisopropylethylamine (55.1 mg, 0.426 mmol) under a nitrogen atmosphere. After 3 h at room temperature the solvent was evaporated and the residue purified by HPL chromatography to yield 34 mg 5-ethyl-2-[5-(4-fluoro-benzenesulfonyl)-4,5,6,7-tetrahydro-1H-pyrazolo[4,3-c]pyridin-3-yl]-7,7-dimethyl-5,7-dihydro-3H-imidazo[4,5-f]indol-6-one (0.0598 mmol, 35%)

MS: M=508.2 (API−)

¹H-NMR (400 MHz, DMSO): δ (ppm)=1.19 (t, 3H), 1.31 (s, 6H), 2.78 (t, 2H), 3.50 (t, 2H), 3.76 (t, 2H), 4.51 (s, 2H), 6.97 and 7.36 (s, 1H, two tautomeric forms), 7.47 (t, 2H), 7.38 and 7.70 (s, 1H, two tautomeric forms), 7.90 (t, 2H), 12.61-12.88 (s, 1H), 12.89-13.19 (s, 1H)

### EXAMPLE 18

### 5-Ethyl-7,7-dimethyl-2-[5-(morpholine-4-carbonyl)-4,5,6,7-tetrahydro-1H-pyrazolo[4,3-c]pyridin-3-yl]-5,7-dihydro-3H-imidazo[4,5-f]indol-6-one

In an analogous manner as described for example 17 5-ethyl-7,7-dimethyl-2-[5-(morpholine-4-carbonyl)-4,5,6,7-tetrahydro-1H-pyrazolo[4,3-c]pyridin-3-yl]-5,7-dihydro-3H-imidazo[4,5-f]indol-6-one was prepared from 5-ethyl-7,7-dimethyl-2-(4,5,6,7-tetrahydro-1H-pyrazolo[4,3-c]pyridin-3-yl)-5,7-dihydro-3H-imidazo[4,5-f]indol-6-one (example 14) and morpholine-4-carbonyl chloride.

MS: M=462.2 (API−)

¹H-NMR (400 MHz, DMSO): δ (ppm)=1.19 (t, 3H), 1.31 (s, 6H), 2.82 (m, 2H), 3.21 (m, 4H), 3.50 (m, 2H), 3.61 (m, 4H), 3.76 (t, 2H), 4.55 (s, 2H), 6.96 and 7.33 (s, 1H, two tautomeric forms), 7.37 and 7.66 (s, 1H, two tautomeric forms), 12.65 and 12.70 (s, 1H, two tautomeric forms), 12.99 and 13.02 (s, 1H, two tautomeric forms)

### EXAMPLE 19

### 2-(5-Benzyl-1,4,5,6-tetrahydro-pyrrolo[3,4-c]pyrazol-3-yl)-5-ethyl-7,7-dimethyl-5,7-dihydro-3H-imidazo[4,5-f]indol-6-one

In an analogous manner as described for example 13 2-(5-benzyl-1,4,5,6-tetrahydro-pyrrolo[3,4-c]pyrazol-3-yl)-5-ethyl-7,7-dimethyl-5,7-dihydro-3H-imidazo[4,5-f]indol-6-one was prepared from 5,6-diamino-1-ethyl-3,3-dimethyl-1,3-dihydro-indol-2-one (A1) and 5-benzyl-1,4,5,6-tetrahydro-pyrrolo[3,4-c]pyrazole-3-carboxylic acid ethyl ester (A9).

MS: M=427.4 (ESI+)

¹H-NMR (400 MHz, DMSO): δ (ppm)=1.09 (t, 3H), 1.29 (s, 6H), 3.74 (m, 2H), 3.87 (m, 4H), 3.99 (s, 2H), 6.96 and 6.25 (s, 1H, two tautomeric forms), 7.27 (m, 1H), 7.42 (m, 4H), 7.37 and 7.60 (s, 1H, two tautomeric forms), 12.60 (br, 1H), 12.95 and 13.25 (br, 1H, two tautomeric forms)

### EXAMPLE 20

### 3-(5-Ethyl-7,7-dimethyl-6-oxo-3,5,6,7-tetrahydro-imidazo[4,5-f]indol-2-yl)-4,6-dihydro-1H-pyrrolo[3,4-c]pyrazole-5-carboxylic acid (2,6-diethyl-phenyl)-amide

To a solution of 5-(2,6-diethyl-phenylcarbamoyl)-1,4,5,6-tetrahydro-pyrrolo[3,4-c]pyrazole-3-carboxylic acid (A11, 230 mg, 0.700 mmol) in absolute DMF (4 ml) under a nitrogen atmosphere were added hydroxybenzotriazole hydrate (128.7 mg, 0.841 mmol), triethylamine (212.6 mg, 293 μl, 2.101 mmol) and N′-(3-dimethylaminopropyl)-N-ethylcarbodiimide hydrochloride (161.1 mg, 0.841 mmol). After 2 h at room temperature a solution of 5,6-diamino-1-ethyl-3,3-dimethyl-1,3-dihydro-indol-2-one (A1, 153.6 mg, 0.700 mmol) in DMF (2 ml) was added and stirring continued at room temperature for 18 h. Most of the DMF was evaporated and the reaction mixture was treated with water. The aqueous phase was extracted twice with ethyl acetate and the solvent of the combined organic phases was evaporated. The residue was dissolved in ethanol (7 ml), treated with HCl (32%, 4 ml) and heated under reflux for 3 h. The solvent was evaporated and the residue alkalized with ammonia (25%). The aqueous phase was extracted three times with ethyl acetate, the combined organic phases were washed with brine, dried over MgSO₄ and the solvent was evaporated. The residue was purified by silica gel chromatography (CH₂Cl₂/MeOH 95:5) to yield 163 mg 3-(5-ethyl-7,7-dimethyl-6-oxo-3,5,6,7-tetrahydro-imidazo[4,5-f]indol-2-yl)-4,6-dihydro-1H-pyrrolo[3,4-c]pyrazole-5-carboxylic acid (2,6-diethyl-phenyl)-amide (0.319 mmol, 45%).

MS: M=512.3 (ESI+)

¹H-NMR (400 MHz, DMSO): δ (ppm)=1.15 (m, 9H), 1.31 (s, 6H), 2.61 (q, 4H), 3.76 (q, 2H), 4.63 (s, 2H), 4.76 (s, 2H), 7.00 an 7.24 (s, 1H, two tautomeric forms), 7.10 (m, 2H), 7.18 (m, 1H), 7.40 and 7.60 (s, 1H, two tautomeric forms), 7.81 (br, 1H), 12.50 and 12.80 (br, 1H, two tautomeric forms), 13.25 and 13.50 (br, 1H, two tautomeric forms)

### EXAMPLE 21

### 3-(5-Isopropyl-7,7-dimethyl-6-oxo-3,5,6,7-tetrahydro-imidazo[4,5-f]indol-2-yl)-4,6-dihydro-1H-pyrrolo[3,4-c]pyrazole-5-carboxylic acid (2,6-diethyl-phenyl)-amide

In an analogous manner as described for example 20 3-(5-Isopropyl-7,7-dimethyl-6-oxo-3,5,6,7-tetrahydro-imidazo[4,5-f]indol-2-yl)-4,6-dihydro-1H-pyrrolo[3,4-c]pyrazole-5-carboxylic acid (2,6-diethyl-phenyl)-amide was prepared from 5-(2,6-diethyl-phenylcarbamoyl)-1,4,5,6-tetrahydro-pyrrolo[3,4-c]pyrazole-3-carboxylic acid (A11) and 5,6-diamino-1-isopropyl-3,3-dimethyl-1,3-dihydro-indol-2-one (A4).

MS: M=526.2 (ESI+)

¹H-NMR (400 MHz, DMSO): δ (ppm)=1.15 (t, 6H), 1.30 (s, 6H), 1.45 (d, 6H), 2.61 (q, 4H), 4.58 (m, 1H), 4.63 (s, 2H), 4.76 (s, 2H), 7.10 (m, 2H), 7.15 (m, 1H), 7.30 and 7.40 (s, 1H, two tautomeric forms), 7.40 and 7.58 (s, 1H, two tautomeris forms), 7.81 (s, 1H), 12.65 andc 12.78 (br, 1H, two tautomeric forms), 13.25 and 13.50 (br, 1H, two tautomeric forms)

### EXAMPLE 22

### 2-(5-Cyclopropanecarbonyl-1,4,5,6-tetrahydro-pyrrolo[3,4-c]pyrazol-3-yl)-5-ethyl-7,7-dimethyl-5,7-dihydro-3H-imidazo[4,5-f]indol-6-one

In an analogous manner as described for example 20 2-(5-cyclopropanecarbonyl-1,4,5,6-tetrahydro-pyrrolo[3,4-c]pyrazol-3-yl)-5-ethyl-7,7-dimethyl-5,7-dihydro-3H-imidazo[4,5-f]indol-6-one was prepared from 5-cyclopropanecarbonyl-1,4,5,6-tetrahydro-pyrrolo[3,4-c]pyrazole-3-carboxylic acid (A12) and 5,6-diamino-1-ethyl-3,3-dimethyl-1,3-dihydro-indol-2-one (A1).

MS: M=405.2 (API+)

¹H-NMR (400 MHz, DMSO): δ (ppm)=0.84 (m, 4H), 1.19 (t, 3H), 1.32 (s, 6H), 1.91 (m, 1H), 3.78 (q, 2H), 4.55 and 4.65 (s, 2H, rotamers), 4.92 and 5.01 (s, 2H, rotamers), 7.05 and 7.26 (s, 1H, two tautomeric forms), 7.45 and 7.65 (s, 1H, two tautomeric forms), 12.75 (br, 1H), 13.30 and 13.55 (br, 1H, two tautomeric forms)

### EXAMPLE 23

### 5-Ethyl-7,7-dimethyl-2-[5-(2-thiophen-2-yl-acetyl)-1,4,5,6-tetrahydro-pyrrolo[3,4-c]pyrazol-3-yl]-5,7-dihydro-3H-imidazo[4,5-f]indol-6-one

In an analogous manner as described for example 20 5-ethyl-7,7-dimethyl-2-[5-(2-thiophen-2-yl-acetyl)-1,4,5,6-tetrahydro-pyrrolo[3,4-c]pyrazol-3-yl]-5,7-dihydro-3H-imidazo[4,5-f]indol-6-one was prepared from 5-(2-thiophen-2-yl-acetyl)-1,4,5,6-tetrahydro-pyrrolo[3,4-c]pyrazole-3-carboxylic acid (A13) and 5,6-diamino-1-ethyl-3,3-dimethyl-1,3-dihydro-indol-2-one (A1).

MS: M=461.1 (ESI+)

¹H-NMR (400 MHz, DMSO): δ (ppm)=1.19 (t, 3H), 1.31 (s, 6H), 3.77 (q, 2H), 4.02 and 4.07 (s, 2H, rotamers), 4.51-4.98 (s, 4H, rotamers), 6.98 (m, 2H), 7.10 and 7.28 (s, 1H, two tautomeric forms), 7.40 (m, 1H), 7.50 and 7.62 (s, 1H, two tautomeric forms), 12.50 and 12.82 (1H, tautomeric forms and rotamers), 13.25-13.55 (1H, tautomeric forms and rotamers)

### EXAMPLE 24

### 5-Isopropyl-7,7-dimethyl-2-[5-(2-thiophen-2-yl-acetyl)-1,4,5,6-tetrahydro-pyrrolo[3,4-c]pyrazol-3-yl]-5,7-dihydro-3H-imidazo[4,5-f]indol-6-one

In an analogous manner as described for example 20 5-Isopropyl-7,7-dimethyl-2-[5-(2-thiophen-2-yl-acetyl)-1,4,5,6-tetrahydro-pyrrolo[3,4-c]pyrazol-3-yl]-5,7-dihydro-3H-imidazo[4,5-f]indol-6-one was prepared from 5-(2-thiophen-2-yl-acetyl)-1,4,5,6-tetrahydro-pyrrolo[3,4-c]pyrazole-3-carboxylic acid (A13) and 5,6-diamino-1-isopropyl-3,3-dimethyl-1,3-dihydro-indol-2-one (A4).

MS: M=475.1 (ESI+)

¹H-NMR (400 MHz, DMSO): δ (ppm)=1.30 (s, 6H), 1.45 (d, 6H), 4.03 and 4.07 (s, 2H, rotamers), 4.51-4.98 (4H, rotamers), 4.58 (m, 1H), 6.98 (m, 2H), 7.10-7.30 (1H, tautomeric forms and rotamers), 7.40 (m, 1H), 7.50-7.65 (1H, tautomeric forms and rotamers), 12.40-12.80 (1H, rotamers and tautomeric forms), 13.20-13.55 (1H, rotamers and tautomeric forms)

### EXAMPLE 25

### 5-Ethyl-2-[5-(4-fluoro-benzenesulfonyl)-1,4,5,6-tetrahydro-pyrrolo[3,4-c]pyrazol-3-yl]-7,7-dimethyl-5,7-dihydro-3H-imidazo[4,5-f]indol-6-one

In an analogous manner as described for example 20 5-ethyl-2-[5-(4-fluoro-benzenesulfonyl)-1,4,5,6-tetrahydro-pyrrolo[3,4-c]pyrazol-3-yl]-7,7-dimethyl-5,7-dihydro-3H-imidazo[4,5-f]indol-6-one was prepared from 5-(4-fluoro-benzenesulfonyl)-1,4,5,6-tetrahydro-pyrrolo[3,4-c]pyrazole-3-carboxylic acid (A14) and 5,6-diamino-1-ethyl-3,3-dimethyl-1,3-dihydro-indol-2-one (A1).

MS: M=495.2 (ESI+)

¹H-NMR (400 MHz, DMSO): δ (ppm)=1.18 (t, 3H), 1.31 (s, 6H), 3.76 (q, 2H), 4.45 (s, 2H), 4.58 (s, 2H), 7.05 and 7.26 (s, 1H, two tautomeric forms), 7.47 (m, 2H), 7.26 and 7.62 (s, 1H, two tautomeric forms), 8.01 (m, 2H), 12.45 and 12.70 (br, 1H, two tautomeric forms), 13.20 and 13.52 (br, 1H, two tautomeric forms)

### EXAMPLE 26

### 5-Ethyl-7,7-dimethyl-2-(1,4,6,7-tetrahydro-thiopyrano[4,3-c]pyrazol-3-yl)-5,7-dihydro-3H-imidazo[4,5-f]indol-6-one

In an analogous manner as described for example 20 5-ethyl-7,7-dimethyl-2-(1,4,6,7-tetrahydro-thiopyrano[4,3-c]pyrazol-3-yl)-5,7-dihydro-3H-imidazo[4,5-f]indol-6-one was prepared from 1,4,6,7-tetrahydro-thiopyrano[4,3-c]pyrazole-3-carboxylic acid (A15) and 5,6-diamino-1-ethyl-3,3-dimethyl-1,3-dihydro-indol-2-one (A1).

MS: M=368.3 (ESI+)

¹H-NMR (400 MHz, DMSO): δ (ppm)=1.19 (t, 3H), 1.30 (s, 6H), 2.92 (s, 4H), 3.75 (m, 2H), 3.99 (s, 2H), 6.97 and 7.29 (s, 1H, two tautomeric forms), 7.37 and 7.63 (s, 1H, two tautomeric forms), 12.63 and 12.68 (br, 1H, two tautomeric forms), 12.96 and 13.00 (br, 1H, two tautomeric forms)

### EXAMPLE 27

### 2-(4,6-Dihydro-1H-thieno[3,4-c]pyrazol-3-yl)-5-ethyl-7,7-dimethyl-5,7-dihydro-3H-imidazo[4,5-f]indol-6-one

In an analogous manner as described for example 20 2-(4,6-dihydro-1H-thieno[3,4-c]pyrazol-3-yl)-5-ethyl-7,7-dimethyl-5,7-dihydro-3H-imidazo[4,5-f]indol-6-one was prepared from 4,6-dihydro-1H-thieno[3,4-c]pyrazole-3-carboxylic acid (A15) and 5,6-diamino-1-ethyl-3,3-dimethyl-1,3-dihydro-indol-2-one (A1).

MS: M=354.2 (ESI+)

¹H-NMR (400 MHz, DMSO): δ (ppm)=1.18 (t, 3H), 1.30 (s, 6H), 3.75 (q, 2H), 4.02 (s, 2H), 4.09 (s, 2H), 6.98 and 7.28 (s, 1H, two tautomeric forms), 7.38 and 7.65 (s, 1H, two tautomeric forms), 12.70 (br, 1H), 13.00 (br, 1H)

### EXAMPLE 28

### 2-(4,6-Dihydro-1H-thieno[3,4-c]pyrazol-3-yl)-5-isopropyl-7,7-dimethyl-5,7-dihydro-3H-imidazo[4,5-f]indol-6-one

In an analogous manner as described for example 20, 2-(4,6-dihydro-1H-thieno[3,4-c]pyrazol-3-yl)-5-isopropyl-7,7-dimethyl-5,7-dihydro-3H-imidazo[4,5-f]indol-6-one was prepared from 4,6-dihydro-1H-thieno[3,4-c]pyrazole-3-carboxylic acid (A15) and 5,6-diamino-1-isopropyl-3,3-dimethyl-1,3-dihydro-indol-2-one (A4).

MS: M=368.1 (ESI+)

¹H-NMR (400 MHz, DMSO): δ (ppm)=1.29 (s, 6H), 1.44 (d, 6H), 4.02 (s, 2H), 4.09 (s, 2H), 4.57 (m, 1H), 7.09 and 7.34 (s, 1H, two tautomeric forms), 7.34 and 7.63 (s, 1H, two tautomeric forms), 12.55 (br, 1H), 13.05 (br, 1H).

Unless stated to the contrary, all compounds in the examples were prepared and characterized as described. All ranges recited herein encompass all combinations and subcombinations included within that range limit. All patents and publications cited herein are hereby incorporated by reference in their entirety for any purpose.

1. A compound according to formula I and all pharmaceutically acceptable salts or esters thereof wherein formula I is: 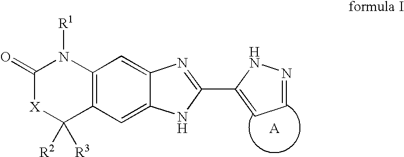
wherein:
(a) R¹ is selected from the group consisting of:
(1) hydrogen;
(2) alkyl, which is optionally substituted one or more times with nitro, cyano or —Y—R⁴;
(3) alkenyl, which is optionally substituted one or more times with nitro, cyano or —Y—R⁴; and
(4) alkynyl, which is optionally substituted one or more times with nitro, cyano or —Y—R⁴;
(b) Y is selected from the group consisting of:
(1) a single bond,
(2) —C(O)NH—;
(3) —C(O)N(alkyl)-;
(4) —N(alkyl)C(O)—;
(5) —NHC(O)—;
(6) —NHC(O)NH—;
(7) —NHC(O)N(alkyl)-;
(8) —NHS(O)₂—;
(9) —S(O)₂NH—;
(10) —S(O)₂N(alkyl)-;
(11) —S(O)₂—;
(12) —S(O)—;
(13) —C(O)O—;
(14) —OC(O)—;
(15) —C(O)—;
(16) —P(O)(alkyl)-;
(17) —NH—;
(18) —N(alkyl)-;
(19) —O—; and
(20) —S—;
(c) R⁴ is selected from the group consisting of:
(1) alkyl, which is optionally substituted one or more times by halogen, hydroxy, alkoxy, alkoxyalkoxy, amino, alkylamino, dialkylamino, —C(O)OH or —C(O)NH₂;
(2) aryl, which is optionally substituted one or morel times by halogen, cyano, nitro, amino, hydroxy, (C₁-C₄)alkyl, (C₁-C₄)alkoxy, halogenated (C₁-C₄)alkyl, or halogenated (C₁-C₄)alkoxy;
(3) heteroaryl, which is optionally substituted one or more times by alkyl;
(4) cycloalkyl; and
(5) heterocyclyl;
(d) R² and R³ form together with the carbon atom to which they are attached a (C₅-C₆)cycloalkyl ring, or alternatively, R² and R³ are independently selected from the group consisting of:
(1) hydrogen; and
(2) alkyl;
(e) X is a single bond, —CH₂— or —C(alkyl)₂—;
(f) ring A is a 5 to 7 membered saturated ring optionally substituted one or more times by alkyl and optionally containing one or two heteroatoms independently selected from the group consisting of oxygen, nitrogen and sulfur, with the remaining ring atoms being carbon atoms, wherein if said ring A contains one nitrogen, said nitrogen can be optionally substituted once by a substituent selected from the group consisting of:
(1) —CH₂-phenyl;
(2) —C(O)-alkyl;
(3) —C(O)-cycloalkyl;
(4) —C(O)-heterocyclyl;
(5) —C(O)—(CH₂)ₙ-phenyl, wherein the phenyl is optionally substituted once or twice with halogen, alkyl, alkoxy, nitro, amino, alkylamino, dialkylamino, cyano, trifluoromethyl or trifluoromethoxy;
(6) —C(O)—(CH₂)ₙ-heteroaryl;
(7) —C(O)—NH-phenyl, wherein the phenyl is optionally substituted once or twice with halogen, alkyl, alkoxy, nitro, amino, alkylamino, dialkylamino, cyano, trifluoromethyl or trifluoromethoxy; and
(8) —S(O)₂-phenyl, wherein the phenyl is optionally substituted once or twice with halogen, alkyl, alkoxy, nitro, amino, alkylamino, dialkylamino, cyano; trifluoromethyl or trifluoromethoxy; and
(g) n is 0, 1 or 2.

2. The compounds according to claim 1, wherein ring A is a 5 to 7 membered saturated ring optionally containing one or two heteroatoms independently selected from the group consisting of oxygen, nitrogen and sulfur, with the remaining ring atoms being carbon atoms, wherein said ring A is optionally substituted one or more times by alkyl.

3. The compounds according to claim 1, wherein:
(a) R¹ is hydrogen or alkyl, wherein said alkyl is optionally substituted once by —Y—R⁴;
(b) Y is a single bond;
(c) R⁴ is heterocyclyl;
(d) R² is alkyl;
(e) R³ is alkyl;
(f) X is a single bond;
(g) wherein if ring A contains one nitrogen, said nitrogen can be optionally substituted once by a substituent selected from the group consisting of:
(1) —CH₂-phenyl,
(2) —C(O)-cycloalkyl,
(3) —C(O)-heterocyclyl,
(4) —C(O)—(CH₂)ₙ-phenyl, wherein the phenyl is optionally substituted once or twice with halogen,
(5) —C(O)—(CH₂)ₙ-heteroaryl,
(6) —C(O)—NH-phenyl, wherein the phenyl is optionally substituted once or twice with alkyl, and
(7) —S(O)₂-phenyl, wherein the phenyl is optionally substituted once or twice with halogen; and
(h) n is 0 or 1.

4. The compounds according to claim 1, wherein:
(a) R¹, R² and R³ are alkyl;
(b) X is a single bond;
(c) ring A is a 5 to 6 membered saturated ring optionally substituted one or more times by alkyl and optionally containing one heteroatom independently selected from the group consisting of oxygen, nitrogen and sulfur, with the remaining ring atoms being carbon atoms, wherein if said ring A contains one nitrogen, said nitrogen can be optionally substituted once by a substitutent selected from the group consisting of:
(1) —CH₂-phenyl,
(2) —C(O)-cycloalkyl,
(3) —C(O)-heterocyclyl,
(4) —C(O)—(CH₂)ₙ-phenyl, wherein the phenyl is optionally substituted once or twice with halogen,
(5) —C(O)—(CH₂)ₙ-thienyl,
(6) —C(O)—NH-phenyl, wherein the phenyl is optionally substituted once or twice with alkyl, and
(7) —S(O)₂-phenyl, wherein the phenyl is optionally substituted once or twice with halogen; and
(d) n is 0 or 1.

5. The compounds according to claim 1, wherein:
(a) R¹ is hydrogen or alkyl, wherein said alkyl is optionally substituted once by —Y—R⁴;
(b) Y is a single bond;
(c) R⁴ is heterocyclyl;
(d) R² is alkyl;
(e) R³ is alkyl;
(f) X is a single bond; and
(g) ring A is a 5 to 7 membered saturated hydrocarbon ring.

6. The compounds according to claim 1, selected from the group consisting of:
7,7-Dimethyl-2-(1,4,5,6-tetrahydro-cyclopentapyrazol-3-yl)-5,7-dihydro-3H-imidazo[4,5-f]indol-6-one;
7,7-Dimethyl-2-(4,5,6,7-tetrahydro-1H-indazol-3-yl)-5,7-dihydro-3H-imidazo[4,5-f]indol-6-one;
5,7,7-Trimethyl-2-(1,4,5,6-tetrahydro-cyclopentapyrazol-3-yl)-5,7-dihydro-3H-imidazo[4,5-f]indol-6-one;
5-Ethyl-7,7-dimethyl-2-(1,4,5,6-tetrahydro-cyclopentapyrazol-3-yl)-5,7-dihydro-3H-imidazo[4,5-f]indol-6-one;
7,7-Dimethyl-5-propyl-2-(1,4,5,6-tetrahydro-cyclopentapyrazol-3-yl)-5,7-dihydro-3H-imidazo[4,5-f]indol-6-one; and
5-Isopropyl-7,7-dimethyl-2-(1,4,5,6-tetrahydro-cyclopentapyrazol-3-yl)-5,7-dihydro-3H-imidazo[4,5-f]indol-6-one.

7. The compounds according to claim 1, selected from the group consisting of:
7,7-Dimethyl-5-(3-morpholin-4-yl-propyl)-2-(1,4,5,6-tetrahydro-cyclopentapyrazol-3-yl)-5,7-dihydro-3H-imidazo[4,5-f]indol-6-one;
5-Ethyl-7,7-dimethyl-2-(4,5,6,7-tetrahydro-1H-indazol-3-yl)-5,7-dihydro-3H-imidazo[4,5-f]indol-6-one;
5-Isopropyl-7,7-dimethyl-2-(4,5,6,7-tetrahydro-1H-indazol-3-yl)-5,7-dihydro-3H-imidazo[4,5-f]indol-6-one;
7,7-Dimethyl-5-(3-morpholin-4-yl-propyl)-2-(4,5,6,7-tetrahydro-1H-indazol-3-yl)-5,7-dihydro-3H-imidazo[4,5-f]indol-6-one; and
5,7,7-Triethyl-2-(4,5,6,7-tetrahydro-1H-indazol-3-yl)-5,7-dihydro-3H-imidazo[4,5-f]indol-6-one.

8. The compounds according to claim 1, wherein:
(a) R¹, R² and R³ are alkyl;
(b) X is a single bond;
(c) ring A contains one heteroatom, wherein if ring A contains nitrogen as the heteroatom, said nitrogen can be optionally substituted once by a substituent selected from the group consisting of:
(1) —CH₂-phenyl,
(2) —C(O)-cycloalkyl,
(3) —C(O)-heterocyclyl,
(4) —C(O)—(CH₂)ₙ-phenyl, wherein the phenyl is optionally substituted once or twice with halogen,
(5) —C(O)—(CH₂)ₙ-thienyl,
(6) —C(O)—NH-phenyl, wherein the phenyl is optionally substituted once or twice with alkyl, and
(7) —S(O)₂-phenyl, wherein the phenyl is optionally substituted once or twice with halogen; and
(d) n is 0 or 1.

9. The compounds according to claim 1, selected from the group consisting of:
5-Ethyl-7,7-dimethyl-2-(1,4,6,7-tetrahydro-pyrano[4,3-c]pyrazol-3-yl)-5,7-dihydro-3H-imidazo[4,5-f]indol-6-one;
2-(5-Benzyl-4,5,6,7-tetrahydro-1H-pyrazolo[4,3-c]pyridin-3-yl)-5-ethyl-7,7-dimethyl-5,7-dihydro-3H-imidazo[4,5-f]indol-6-one;
5-Ethyl-7,7-dimethyl-2-(4,5,6,7-tetrahydro-1H-pyrazolo[4,3-c]pyridin-3-yl)-5,7-dihydro-3H-imidazo[4,5-f]indol-6-one;
2-(5-Benzyl-1,4,5,6-tetrahydro-pyrrolo[3,4-c]pyrazol-3-yl)-5-ethyl-7,7-dimethyl-5,7-dihydro-3H-imidazo[4,5-f]indol-6-one;
5-Ethyl-7,7-dimethyl-2-(1,4,6,7-tetrahydro-thiopyrano[4,3-c]pyrazol-3-yl)-5,7-dihydro-3H-imidazo[4,5-f]indol-6-one; and
2-(5-Cyclopropanecarbonyl-1,4,5,6-tetrahydro-pyrrolo[3,4-c]pyrazol-3-yl)-5-ethyl-7,7-dimethyl-5,7-dihydro-3H-imidazo[4,5-f]indol-6-one.

10. The compounds according to claim 1, selected from the group consisting of:
5-Ethyl-2-{5-[2-(4-fluoro-phenyl)-acetyl]-4,5,6,7-tetrahydro-1H-pyrazolo[4,3-c]pyridin-3-yl}-7,7-dimethyl-5,7-dihydro-3H-imidazo[4,5-f]indol-6-one;
5-Ethyl-2-[5-(4-fluoro-benzenesulfonyl)-4,5,6,7-tetrahydro-1H-pyrazolo[4,3-c]pyridin-3-yl]-7,7-dimethyl-5,7-dihydro-3H-imidazo[4,5-f]indol-6-one;
5-Ethyl-7,7-dimethyl-2-[5-(morpholine-4-carbonyl)-4,5,6,7-tetrahydro-1H-pyrazolo[4,3-c]pyridin-3-yl]-5,7-dihydro-3H-imidazo[4,5-f]indol-6-one;
5-Ethyl-2-[5-(4-fluoro-benzoyl)-4,5,6,7-tetrahydro-1H-pyrazolo[4,3-c]pyridin-3-yl]-7,7-dimethyl-5,7-dihydro-3H-imidazo[4,5-f]indol-6-one;
2-(4,6-Dihydro-1H-thieno[3,4-c]pyrazol-3-yl)-5-ethyl-7,7-dimethyl-5,7-dihydro-3H-imidazo[4,5-f]indol-6-one; and
5-Ethyl-7,7-dimethyl-2-[5-(2-thiophen-2-yl-acetyl)-1,4,5,6-tetrahydro-pyrrolo[3,4-c]pyrazol-3-yl]-5,7-dihydro-3H-imidazo[4,5-f]indol-6-one.

11. The compounds according to claim 1, selected from the group consisting of:
2-(4,6-Dihydro-1H-thieno[3,4-c]pyrazol-3-yl)-5-isopropyl-7,7-dimethyl-5,7-dihydro-3H-imidazo[4,5-f]indol-6-one;
3-(5-Ethyl-7,7-dimethyl-6-oxo-3,5,6,7-tetrahydro-imidazo[4,5-f]indol-2-yl)-4,6-dihydro-1H-pyrrolo[3,4-c]pyrazole-5-carboxylic acid (2,6-diethyl-phenyl)-amide;
5-Ethyl-2-[5-(4-fluoro-benzenesulfonyl)-1,4,5,6-tetrahydro-pyrrolo[3,4-c]pyrazol-3-yl]-7,7-dimethyl-5,7-dihydro-3H-imidazo[4,5-f]indol-6-one;
3-(5-Isopropyl-7,7-dimethyl-6-oxo-3,5,6,7-tetrahydro-imidazo[4,5-f]indol-2-yl)-4,6-dihydro-1H-pyrrolo[3,4-c]pyrazole-5-carboxylic acid (2,6-diethyl-phenyl)-amide; and
5-Isopropyl-7,7-dimethyl-2-[5-(2-thiophen-2-yl-acetyl)-1,4,5,6-tetrahydro-pyrrolo[3,4-c]pyrazol-3-yl]-5,7-dihydro-3H-imidazo[4,5-f]indol-6-one.

12. The compounds according to claim 1, wherein:
(a) R¹, R² and R³ are alkyl;
(b) X is a single bond; and
(c) ring A is a 5 to 7 membered saturated ring containing one heteroatom independently selected from oxygen, nitrogen or sulfur and the remaining ring atoms being carbon atoms.

13. The compounds according to claim 1, wherein:
(a) R¹, R² and R³ are alkyl;
(b) X is a single bond; and
(c) ring A contains one nitrogen which is substituted by a substituent selected from the group consisting of:
(1) —CH₂-phenyl,
(2) —C(O)-alkyl,
(3) —C(O)-cycloalkyl,
(4) —C(O)-heterocyclyl,
(5) —C(O)—(CH₂)ₙ-phenyl, wherein the phenyl is optionally substituted once or twice with halogen, alkyl, alkoxy, nitro, amino, alkylamino, dialkylamino, cyano, trifluoromethyl or trifluoromethoxy,
(6) —C(O)—(CH₂)ₙ-heteroaryl,
(7) —C(O)—NH-phenyl, wherein the phenyl is optionally substituted once or twice with halogen, alkyl, alkoxy, nitro, amino, alkylamino, dialkylamino, cyano, trifluoromethyl or trifluoromethoxy, and
(8) —S(O)₂-phenyl, wherein the phenyl is optionally substituted once or twice with halogen, alkyl, alkoxy, nitro, amino, alkylamino, dialkylamino, cyano, trifluoromethyl or trifluoromethoxy; and
n is 0, 1 or 2.

14. The compounds according to claim 1, wherein:
(a) R¹, R² and R³ are alkyl;
(b) X is a single bond;
(c) ring A contains one nitrogen which is substituted once by a substituent selected from the group consisting of:
(1) —CH₂-phenyl,
(2) —C(O)-cycloalkyl,
(3) —C(O)-heterocyclyl,
(4) —C(O)—(CH₂)ₙ-phenyl, wherein the phenyl is optionally substituted once or twice with halogen,
(5) —C(O)—(CH₂)ₙ-thienyl,
(6) —C(O)—NH-phenyl, wherein the phenyl is optionally substituted once or twice with alkyl, and
(7) —S(O)₂-phenyl, wherein the phenyl is optionally substituted once or twice with halogen; and
(d) n is 0 or 1.

15. A process for the preparation of the compounds of formula I in claim 1 comprising the steps of:
(a) reacting a compound of formula II: 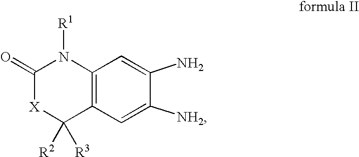
wherein X, R¹, R² and R³ are defined in claim 1,
with a compound of formula III: 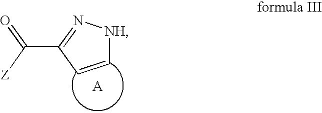
wherein ring A is defined in claim 1 and Z is selected from the group consisting of —OH, —Cl, —H, —OMe and hydroxybenzotriazole;
to obtain the compounds of formula I: 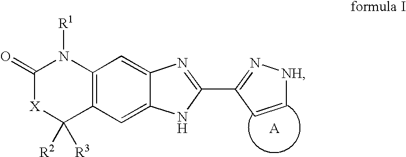
(b) optionally isolating the compounds of formula; and
(c) optionally converting the compounds of formula I into their pharmaceutically acceptable salts or esters.

16. A pharmaceutical composition comprising a therapeutically effective amount of a compound of claim 1 and a pharmaceutically acceptable carrier.

17. A method of preventing or treating a disease or condition mediated by an inappropriate activation of Aurora family tyrosine kinases comprising administering to a person in need thereof a therapeutically effective amount of a compound of claim 1.

18. The method of claim 17 wherein the disease or condition is tumor growth.

19. The method of claim 17 wherein the disease or condition is colorectal cancer, breast cancer, lung cancer, prostate cancer, pancreatic cancer, gastric cancer, bladder cancer, ovarian cancer, melanoma, neuroblastoma, cervical cancer, renal cancer, leukemia or lymphoma.

20. The method of claim 17 wherein the disease or condition is acute-myelogenous leukemia acute lymphocytic leukemia, or gastrointestinal stromal tumor.
